(19) United States
(12) Patent Application Publication
(10) Pub. No.: US 2022/0089631 A1
(43) Pub. Date: Mar. 24, 2022

(54) COMPOUND COMPRISING MULTIVALENT SIALYLOLIGOSACCHARIDE RESIDUES, AND COMPOSITION FOR PREVENTING OR TREATING VIRAL INFECTION DISEASES, CONTAINING SAME AS ACTIVE INGREDIENT

(71) Applicant: Genechem Inc., Daejeon (KR)

(72) Inventors: Jin Suk WOO, Daejeon (KR); Dae Hee KIM, Daejeon (KR); Sang Mi LEE, Daejeon (KR); Sang Eun YOO, Daejeon (KR); Won Min SEO, Sejong-si (KR); Ji Young YANG, Daejeon (KR); Li La KIM, Chungcheongbuk-do (KR)

(21) Appl. No.: 17/270,184

(22) PCT Filed: Aug. 22, 2019
(86) PCT No.: PCT/KR2019/010716

§ 371 Date: Feb. 22, 2021

(30) Foreign Application Priority Data
Aug. 23, 2018 (KR) 10-2018-0098728
Aug. 22, 2019 (KR) 10-2019-0103068

(51) Int. Cl. C07H 17/04; C07H 15/18; A61K 9/00; A61K 9/48; A61K 9/20; A61P 31/16

(52) U.S. Cl. CPC C07H 17/04; C07H 15/18; A61K 9/0095; A61K 9/0056; A61K 9/0019; A61P 31/16; A61K 9/4866; A61K 9/4825; A61K 9/2018; A61K 9/2059; A61K 9/4858

(57) ABSTRACT

The present invention relates to a use of a compound comprising a multivalent sialyloligosaccharide residue, an optical isomer thereof or a pharmaceutically acceptable salt thereof, and a composition comprising the same as an active ingredient for preventing or treating viral infectious diseases. The present invention shows high hemagglutination inhibitory activity and virus neutralizing activity so as to have excellent antiviral activity, thereby being effectively usable as a composition for preventing or treating viral infectious diseases.

### TECHNICAL FIELD

The present invention relates to antiviral activity of a compound comprising a multivalent sialyloligosaccharide residue, an optical isomer thereof, or a pharmaceutically acceptable salt thereof.

This application claims priority to and the benefit of Korean Patent Application Nos. 10-2018-0098728 and 10-2019-0103068 filed in the Korean Intellectual Property Office on Aug. 23, 2018 and Aug. 22, 2019, respectively, and all the contents disclosed in the specification and drawings of those applications are incorporated in this application.

### BACKGROUND ART

Most of the diseases that have hit the world over the last few decades are viral diseases, and for example, influenza viruses such as H7N9 or H5N6 that broke out in China, Ebola virus that broke out in West Africa, and Zika virus that broke out in Mexico and South America correspond to the viral diseases. These diseases have imposed a huge financial burden and threatened human health internationally, various vaccines and new drugs have been developed to control the outbreak of viral diseases, but viruses have become resistant to medicines while constantly changing to new types, so that the mutation rates of virus have not been able to be suppressed. Therefore, there is a need for solving problems by a new conceptual method different from existing methods.

Among the viruses mentioned above, influenza viruses do not infect most humans, but easily infect birds and has caused serious economic damage to the world over several years. The H9N2 type virus found in the Republic of Korea has infected the entire South Korean region and has caused great damage such as decreased egg production and the death of poultry. To control these viruses, many countries have even implemented vaccine production and new drug development, high levels of biological isolation and control of the movement of poultry, personnel and vehicles. Among them, vaccine production for livestock is the most effective method for preventing and controlling the spread of influenza. However, since the genetic flexibility of the virus has always been a threat to humans, vaccines should always be prepared for the emergence of a new species of viruses. The main problem for this is that the development period for many medicines is longer than the viral adaptation and resistance period.

The mechanism of virus adsorption to host cells is an important part in the infection mechanism (Chandrasekaran A, et al, Nat Biotechnol, 26, 107-113, 2008). The blocking and preventing of the adsorption of this pathogen to the host cell surface would be a simple and clear method for removing the pathogen from a host (Salcedo J, et al, Food Chem, 136, 726-734, 2013).

Meanwhile, as a related art document related to a composition for treating viral infectious diseases, including a compound comprising a sialyloligosaccharide, a related art document [Johansson S M, et al, Chembiochem, 6, 358-364, 2005] discloses that human serum albumin (HSA) including multivalent 3′-sialyl lactose can be used as an antiviral drug for epidemic keratoconjunctivitis due to its adenovirus (Ad37) infection inhibitory activity.

However, as in the present invention, there has been no previous report confirming that antiviral activity is excellent by selecting a multivalent sialyloligosaccharide including a sugar having a cycloalkyl structure as a core structure.

### DISCLOSURE

### Technical Problem

An object of the present invention is to provide a compound comprising a multivalent sialyloligosaccharide residue, an optical isomer thereof or a pharmaceutically acceptable salt thereof, and a composition, an animal drug, an animal feed additive, or a quasi-drug composition comprising the same as an active ingredient for preventing or treating viral infectious diseases.

### Technical Solution

To solve the above-described problem, the present invention provides a compound comprising a multivalent sialyloligosaccharide residue, which is represented by the following Chemical Formula 1, an optical isomer thereof, or a pharmaceutically acceptable salt thereof.

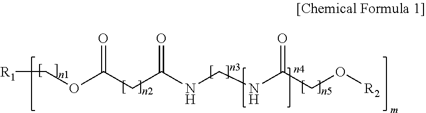

In Chemical Formula 1,

R₁ is a C₄-C₆ cycloalkyl or a C₄-C₆ heterocycloalkyl,

R₂ is a sialyloligosaccharide,

n₁ is 0 or 1, n₂ is 1 or 2, n3 is an integer from 2 to 10, n₄ is 0 or 1, and n₅ is an integer from 1 to 8, and

m is an integer from 1 to 6. Preferably, m is an integer from 2 to 6.

A specific aspect provides a compound comprising a multivalent sialyloligosaccharide residue, an optical isomer thereof, or a pharmaceutically acceptable salt thereof, wherein

in Chemical Formula 1,

R₁ is a C₄-C₆ cycloalkyl or a C₄-C₆ heterocycloalkyl,

R₂ is a sialyl lactose,

n₁ is 0 or 1, n₂ is 1 or 2, n₃ is an integer from 2 to 10, n₄ is 0 or 1, and n₅ is an integer from 1 to 8, where the sum of n₃ and n₅ is an integer from 4 to 8, and

m is an integer from 4 to 6.

A specific aspect provides a compound comprising a multivalent sialyloligosaccharide residue, an optical isomer thereof, or a pharmaceutically acceptable salt thereof, wherein

in Chemical Formula 1,

R₁ is a C₄ or C₆ cycloalkyl,

R₂ is a sialyl lactose,

n₁ is 0 or 1, n₂ is 1 or 2, n₃ is an integer from 2 to 10, n₄ is 0 or 1, and n₅ is an integer from 1 to 8, where the sum of n₃ and n₅ is an integer of 6 or 7, and

m is an integer of 4 or 6.

A specific aspect provides a compound comprising a multivalent sialyloligosaccharide residue, which is selected from the group consisting of the following structures of Chemical Formulae 2 to 7, an optical isomer thereof, or a pharmaceutically acceptable salt thereof, wherein:

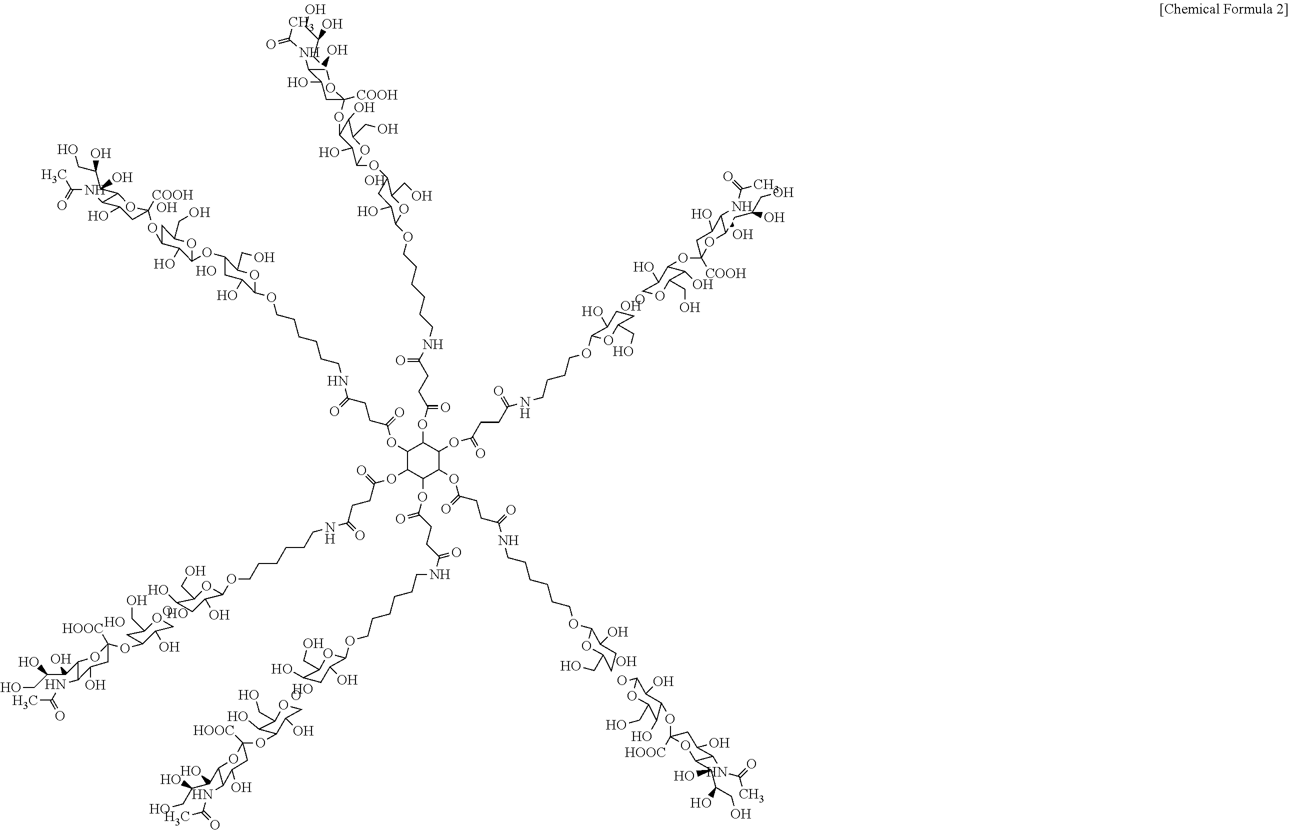

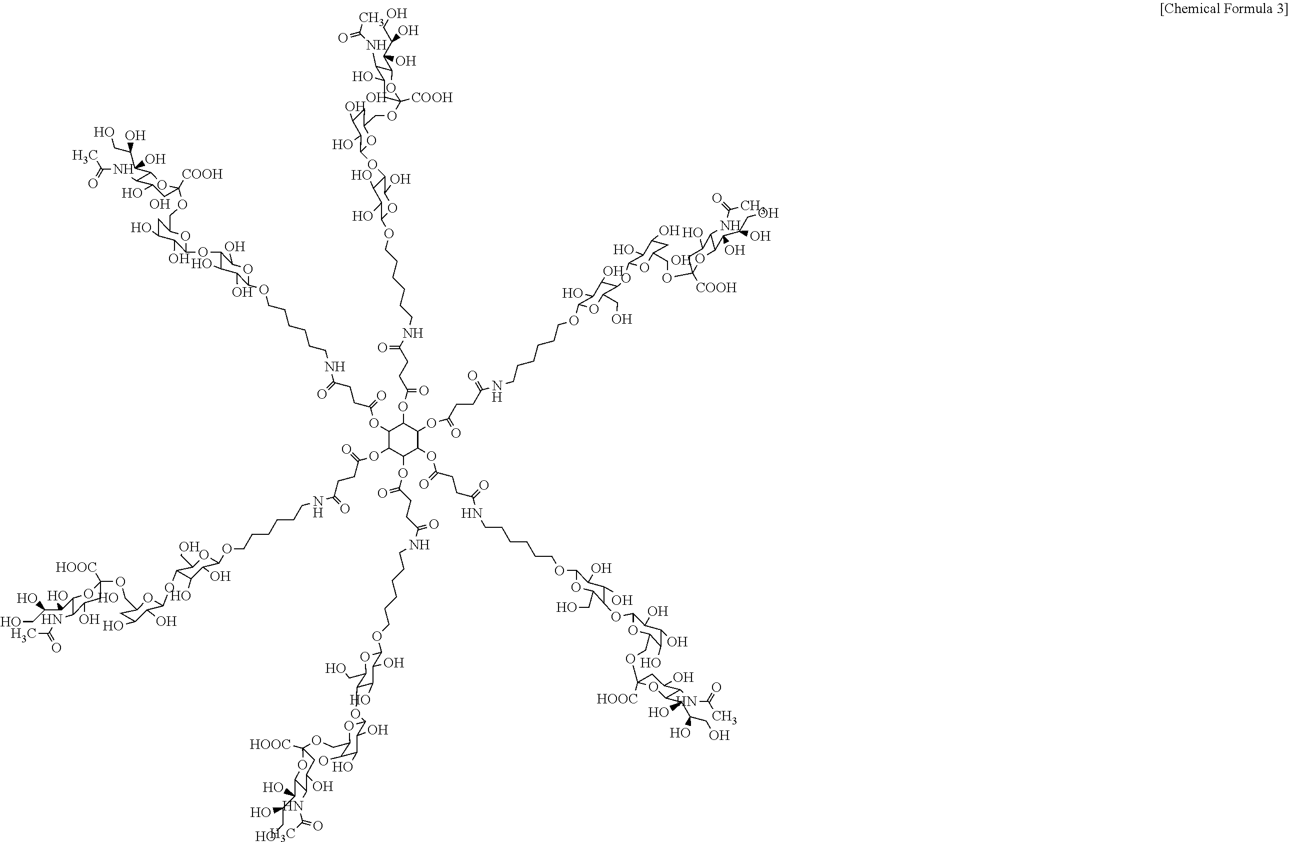

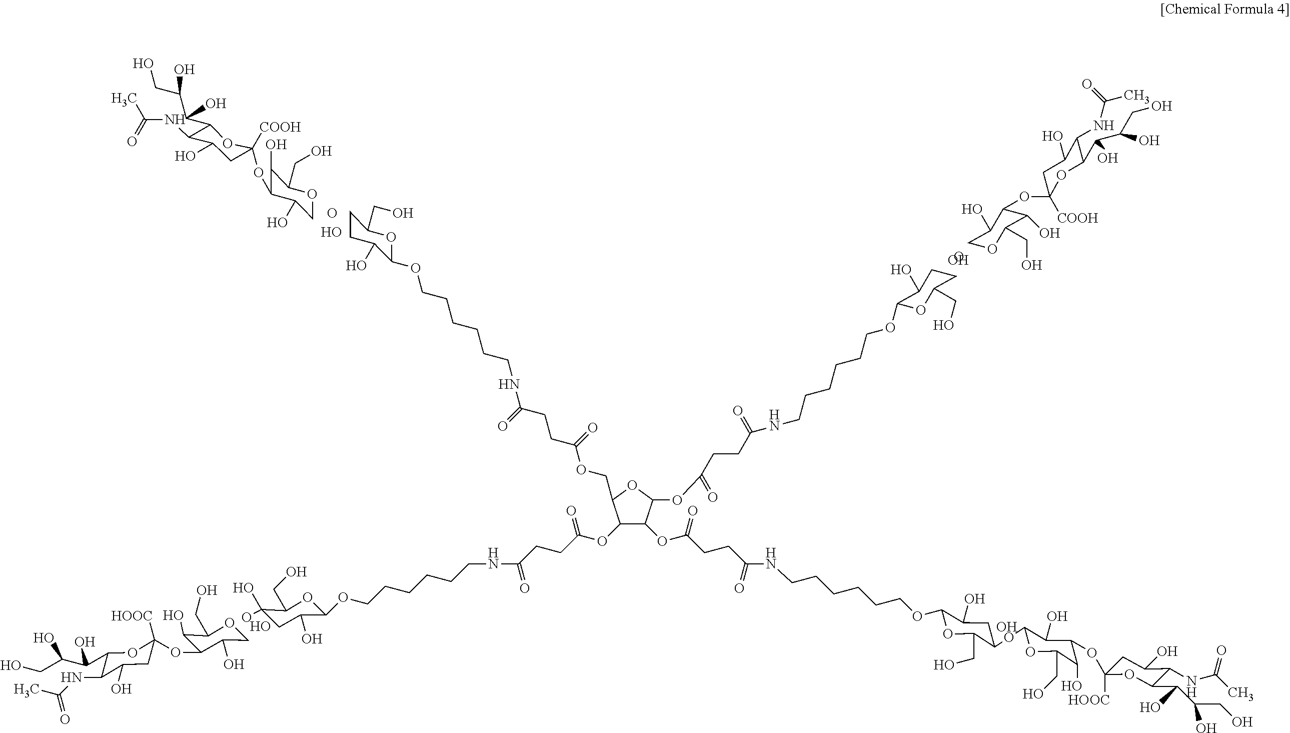

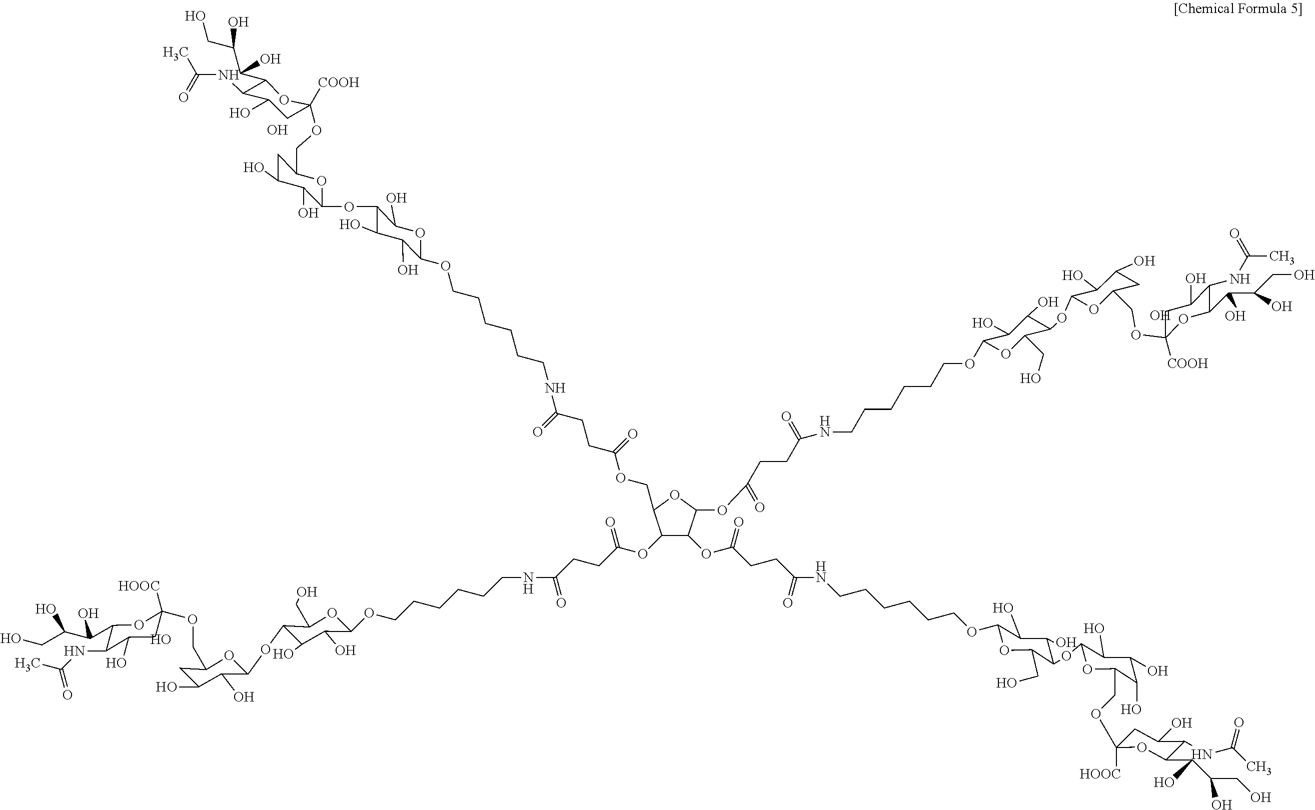

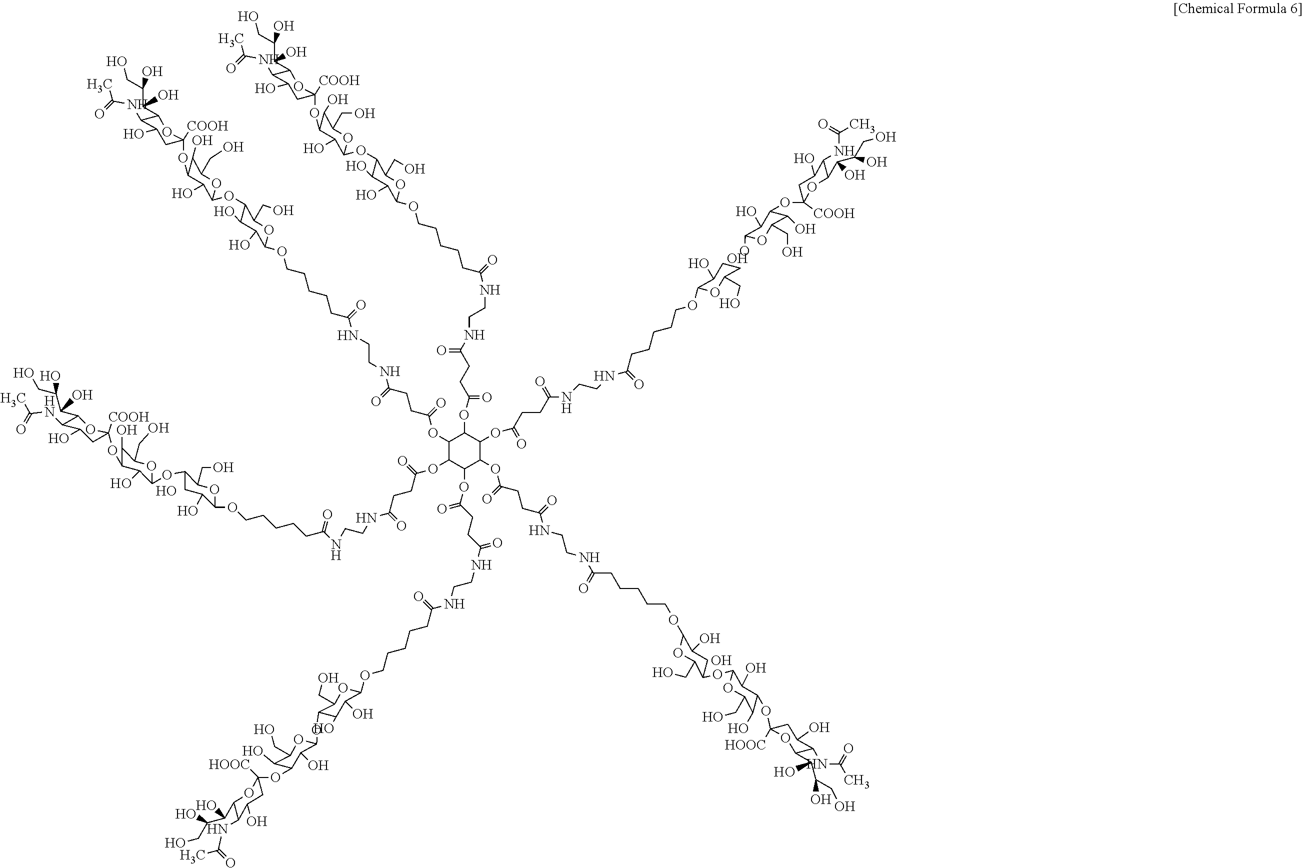

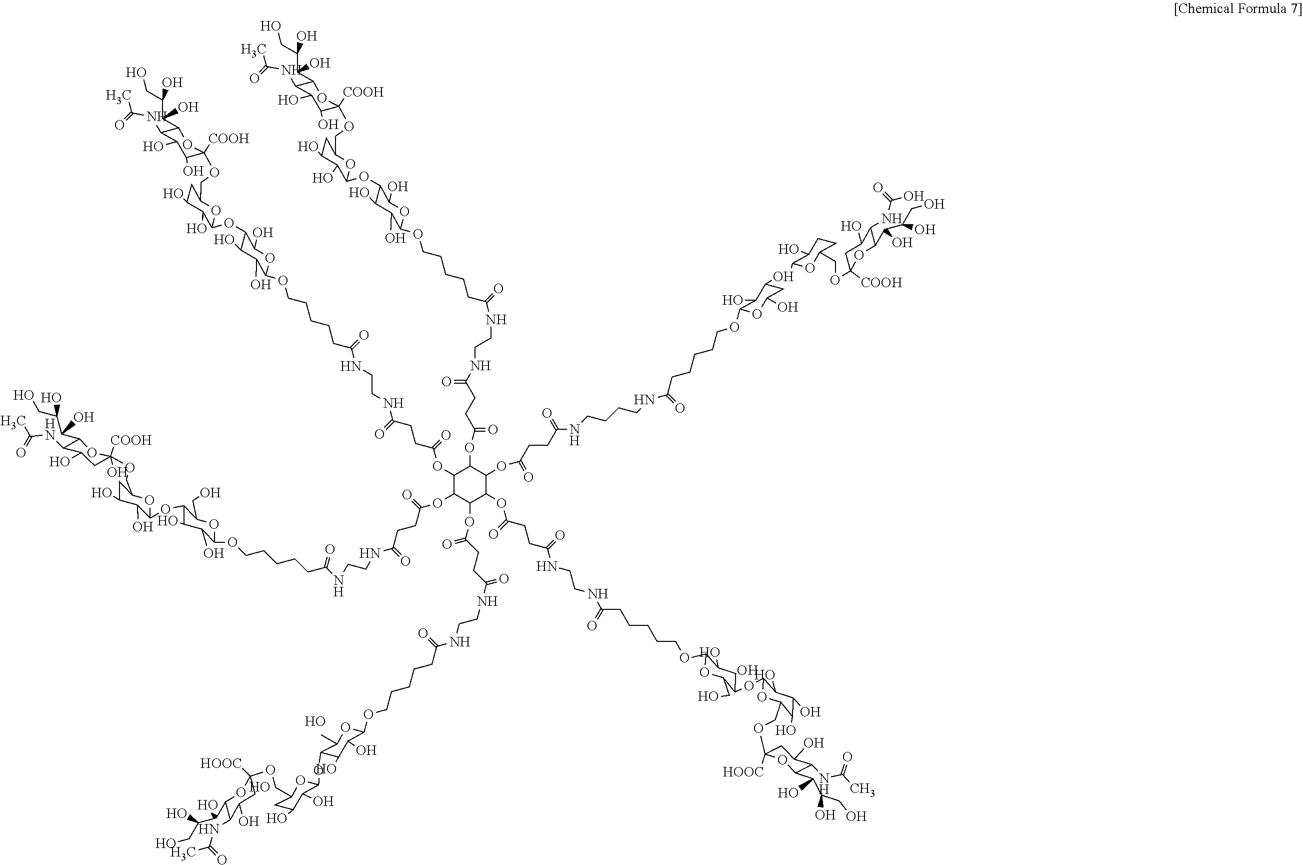

Further, the present invention provides a pharmaceutical composition for preventing or treating viral infectious diseases, an animal drug for preventing or treating viral infectious diseases, a health functional food for preventing or ameliorating viral infectious diseases, an animal feed additive for preventing or ameliorating viral infectious diseases, and a quasi-drug composition for preventing or ameliorating viral infectious diseases, comprising a multivalent sialyloligosaccharide residue, an optical isomer thereof, or a pharmaceutically acceptable salt thereof as an active ingredient.

The present invention provides a method for treating viral infectious diseases of an individual in need thereof, the method comprising: administering a therapeutically effective amount of a compound comprising a multivalent sialyloligosaccharide residue, which is represented by Chemical Formula 1, an optical isomer thereof or a pharmaceutically acceptable salt thereof to an individual.

In addition, the present invention provides a use of a composition comprising a compound comprising a multivalent sialyloligosaccharide residue, which is represented by Chemical Formula 1, an optical isomer thereof, or a pharmaceutically acceptable salt thereof as an active ingredient for preventing, ameliorating, or treating viral diseases.

Preferably, the virus may be an influenza virus.

More preferably, the influenza virus may be influenza virus type A or influenza virus type B.

Still more preferably, the influenza virus type A may be one or more selected from the group consisting of H1N1, H3N2, and H9N2.

Furthermore, the viral infectious disease may be the flu, the common cold, pharyngolaryngitis, bronchitis, pneumonia, avian flu, or swine flu.

### Advantageous Effects

The present invention relates to a use of a composition compound comprising a multivalent sialyloligosaccharide residue, an optical isomer thereof or a pharmaceutically acceptable salt thereof as an active ingredient by utilizing the antiviral activity of the same for preventing, ameliorating, or treating viral infectious diseases. The present invention shows high hemagglutination inhibitory activity and virus neutralizing activity so as to have excellent antiviral activity, thereby being effectively usable for preventing, ameliorating, or treating viral infectious diseases.

### DESCRIPTION OF DRAWINGS

FIG. 1 illustrates a structural formula of Chemical Formula 1 of a compound of the present invention.

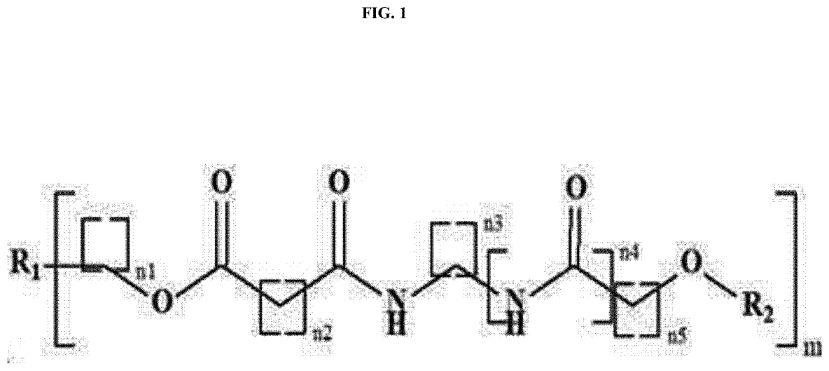

FIG. 2 is a graph of the MALDI-TOF measurement results of Compound 1.

FIG. 3 is a graph of the MALDI-TOF measurement results of Compound 2.

FIG. 4 is a graph of the MALDI-TOF measurement results of Compound 3.

FIG. 5 is a graph of the MALDI-TOF measurement results of Compound 4.

FIG. 6 is a graph of the NMR measurement results of Compound 5.

FIG. 7 is a graph of the NMR measurement results of Compound 6.

FIG. 8 illustrates the results of confirming the H1N1 virus neutralizing activity of Compound 1 and Compound 2 under a fluorescence microscope.

### MODES OF THE INVENTION

Since the present invention may be modified into various forms and include various exemplary embodiments, specific exemplary embodiments will be illustrated in the drawings and described in detail in the Detailed Description. However, the description is not intended to limit the present invention to the specific exemplary embodiments, and it is to be understood that all the changes, equivalents, and substitutions belonging to the spirit and technical scope of the present invention are included in the present invention. When it is determined that the detailed description of the related publicly known art in describing the present invention may obscure the gist of the present invention, the detailed description thereof will be omitted.

Hereinafter, the present invention will be described in detail.

The present invention relates to a compound comprising a multivalent sialyloligosaccharide residue, which is represented by the following Chemical Formula 1, an optical isomer thereof, or a pharmaceutically acceptable salt thereof

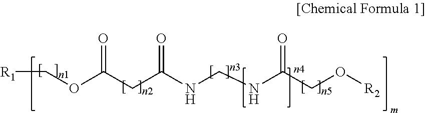

In Chemical Formula 1,

R₁ is a C₄-C₆ cycloalkyl or a C₄-C₆ heterocycloalkyl,

R₂ is a sialyloligosaccharide,

n₁ is 0 or 1, n₂ is 1 or 2, n₃ is an integer from 2 to 10, n₄ is 0 or 1, and n₅ is an integer from 1 to 8, and

m is an integer from 1 to 6. Preferably, m is an integer from 2 to 6.

Preferably, in Chemical Formula 1,

R₁ is a C₄-C₆ cycloalkyl or a C₄-C₆ heterocycloalkyl,

R₂ is a sialyl lactose,

n₁ is 0 or 1, n₂ is 1 or 2, n₃ is an integer from 2 to 10, n₄ is 0 or 1, and n₅ is an integer from 1 to 8, where the sum of n₃ and n₅ is an integer from 4 to 8, and

m is an integer from 4 to 6.

More preferably, in Chemical Formula 1,

R₁ is a C₄ or C₆ cycloalkyl,

R₂ is a sialyl lactose,

n₁ is 0 or 1, n₂ is 1 or 2, n₃ is an integer from 2 to 10, n₄ is 0 or 1, and n₅ is an integer from 1 to 8, where the sum of n₃ and n₅ is an integer of 6 or 7, and

m is an integer of 4 or 6.

The term “cycloalkyl” refers to a monocyclic, saturated hydrocarbon group, and includes cyclobutyl, cyclopentyl, cyclohexyl, and the like depending on the number of carbon atoms.

The term “heterocycloalkyl” refers to a monocyclic, saturated hydrocarbon group including one or more heteroatoms, such as N, O, or S, and includes aziridine, pyrrolidine, piperidine, piperazine, morpholine, tetrahydrofuran, tetrahydropyran, and the like, depending on the number and type of heteroatoms and the number of carbon atoms included in the ring.

The term “sialyloligosaccharide” is an oligosaccharide having sialic acid at a non-reducing end, is mainly present in mammalian milk, and is also contained in urine in a small amount. Sialyl lactose is included in sialyloligosaccharides, and sialyl lactose has two structural isomers, 3′-sialyl lactose and 6′-sialyl lactose. Sialyloligosaccharides can be obtained by various methods, can be prepared by using commercially available products, or can be directly prepared, and in regard to the preparation method, the methods described in Korean Patent Nos. 10-1560311, 10-1574952, and 10-088665 filed by the applicant of the present invention may be referenced and applied.

When more specifically illustrated, the compound of Chemical Formula 1 of the present invention has a structure as shown in the following Chemical Formulae 2 to 7 (Compounds 1 to 6).

Hereinafter, Compound 1 of Chemical Formula 2 is also referred to as Hex-AH-3′SL.

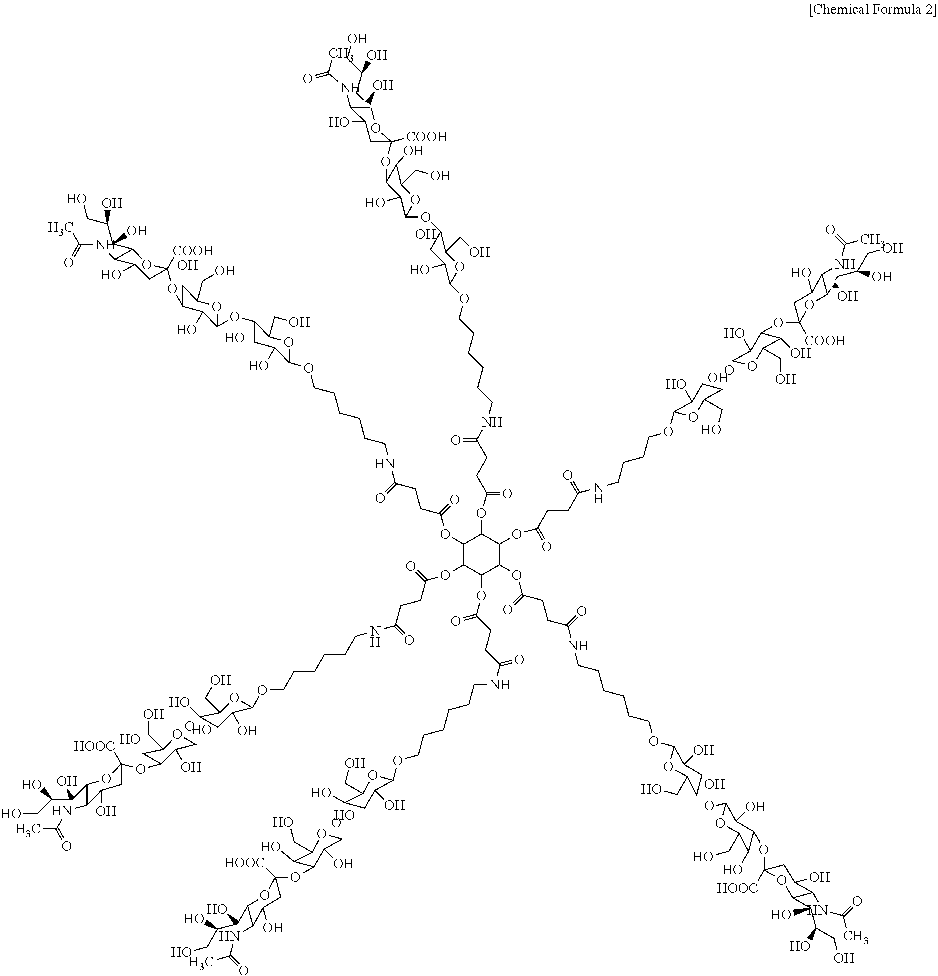

Hereinafter, Compound 2 of Chemical Formula 3 is also referred to as Hex-AH-6′SL.

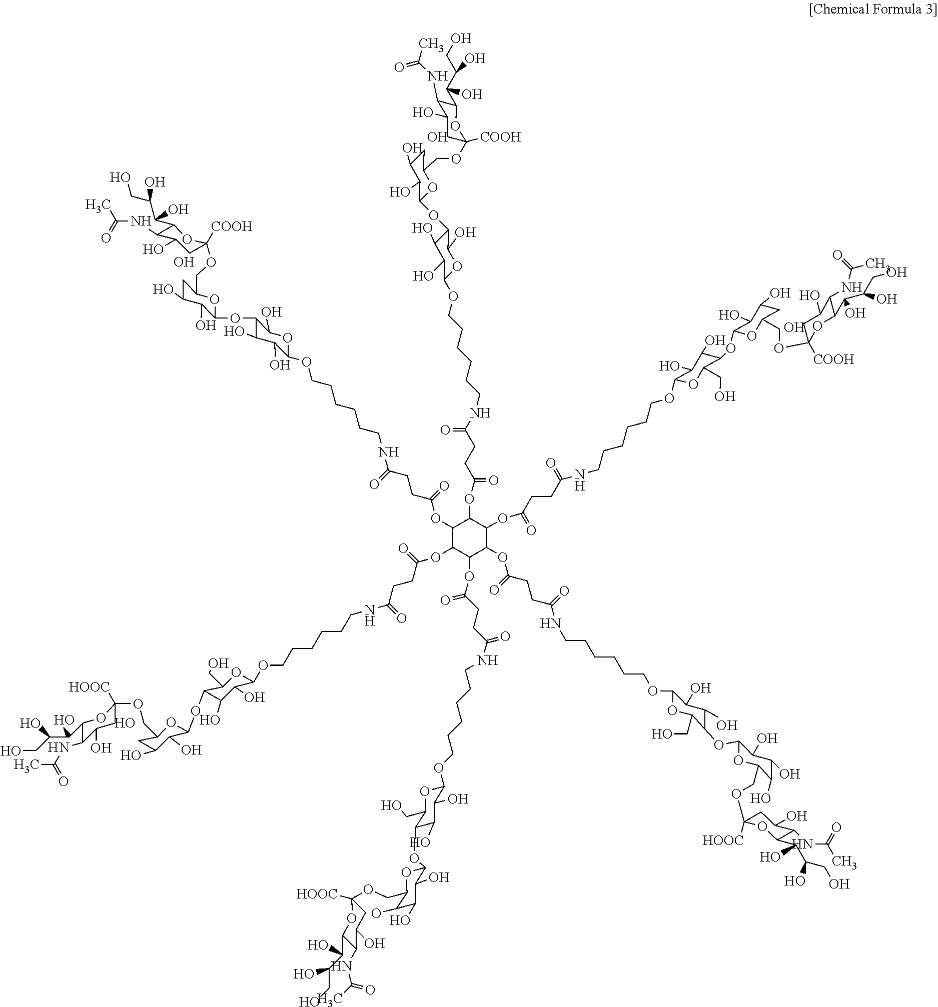

Hereinafter, Compound 3 of Chemical Formula 4 is also referred to as Tetra-AH-3′SL.

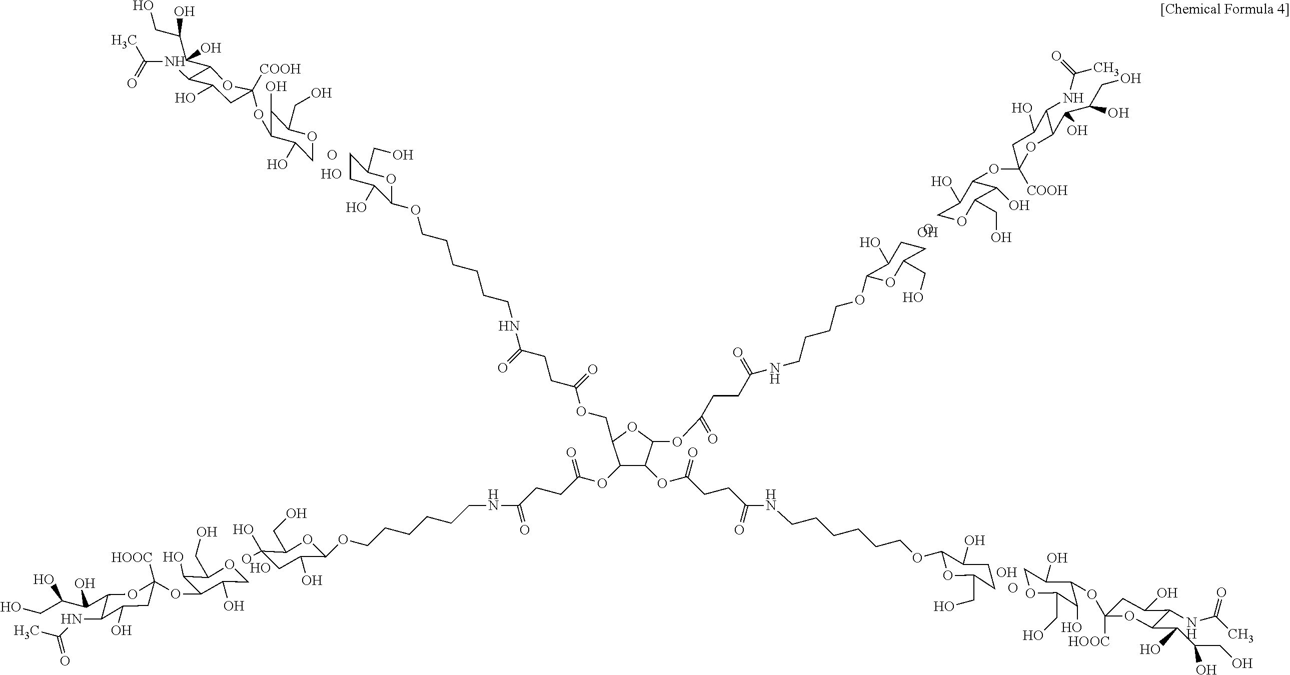

Hereinafter, Compound 4 of Chemical Formula 5 is also referred to as Tetra-AH-6′SL.

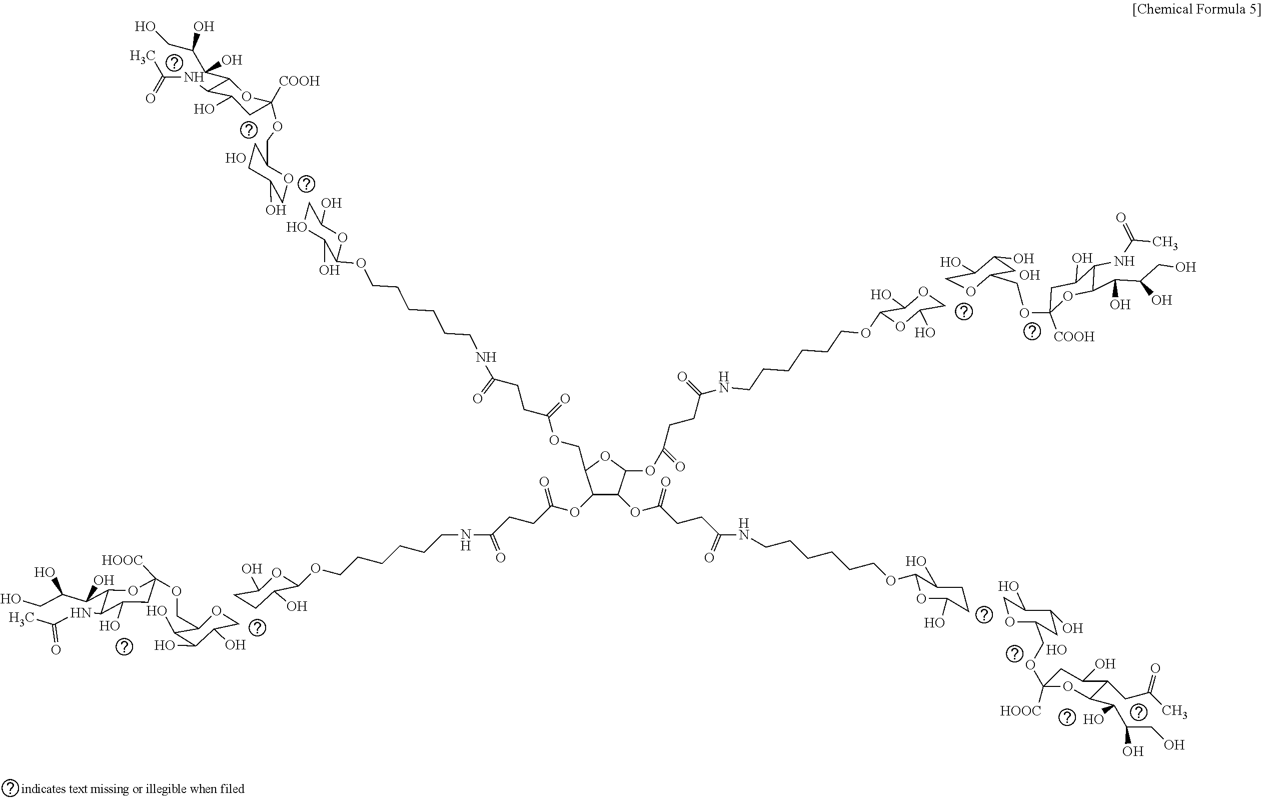

Hereinafter, Compound 5 of Chemical Formula 6 is also referred to as Hex-Carboxyl-6′SL.

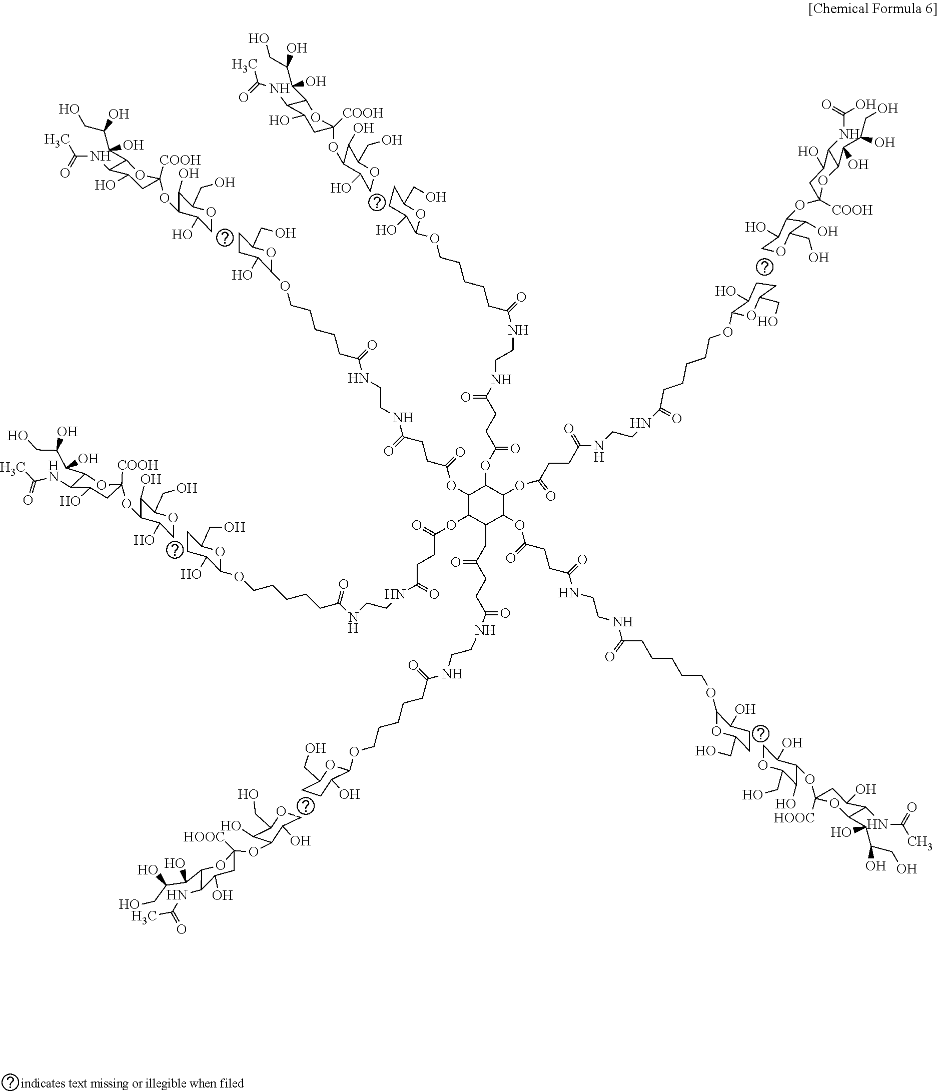

Hereinafter, Compound 6 of Chemical Formula 7 is also referred to as Hex-Carboxyl-3′SL.

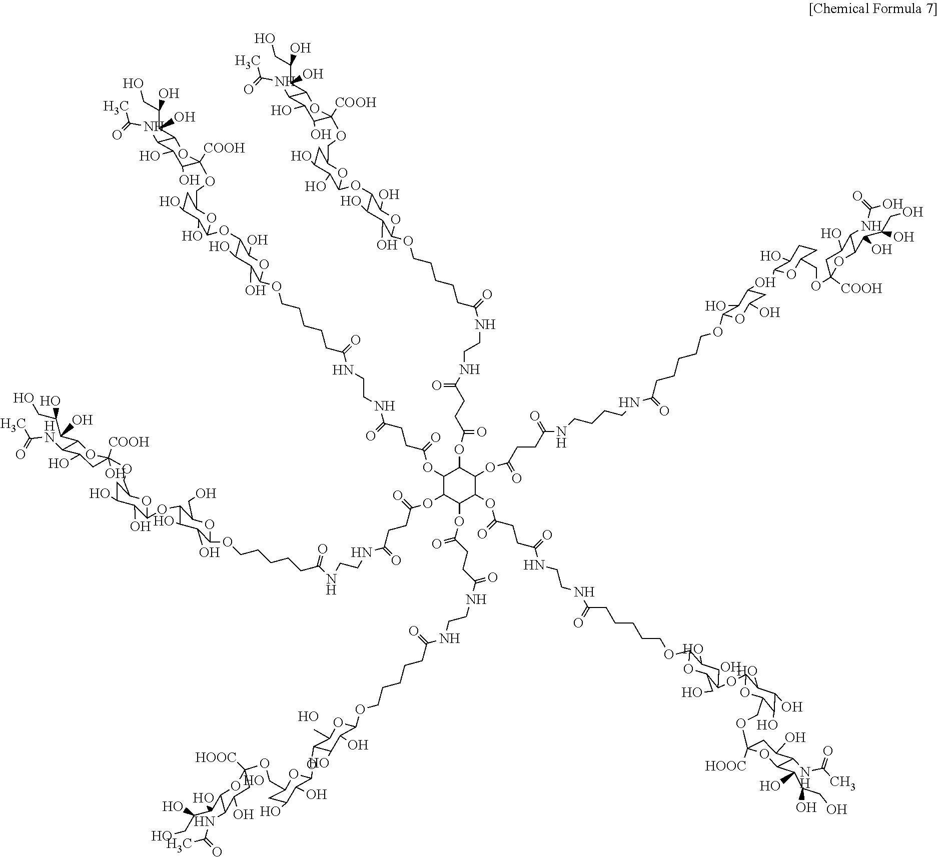

In the present invention, the term “optical isomer” includes R-forms, S-forms, or the form of a racemic compound, respectively.

Examples of a pharmaceutically acceptable salt of the compound comprising a multivalent sialyloligosaccharide residue of the present invention include addition salts formed by an inorganic acid, such as hydrochlorides, sulfates, phosphates, hydrobromides, hydroiodides, nitrates, pyrosulfates, and metaphosphates, addition salts formed by an organic acid, such as citrates, oxalates, benzoates, acetates, trifluoroacetates, propionates, succinates, fumarates, lactates, maleates, tartrates, glutarates, methane sulfonates, toluene sulfonates, and sulfonates, or metal salts such as lithium salts, sodium salts, potassium salts, magnesium salts, and calcium salts, but are not limited thereto.

In addition, the present invention provides a composition comprising a compound comprising a multivalent sialyloligosaccharide residue, which is represented by Chemical Formula 1, an optical isomer thereof, or a pharmaceutically acceptable salt thereof as an active ingredient for preventing, ameliorating, or treating viral infectious diseases.

Further, the present invention provides a method for treating viral infectious diseases of an individual in need thereof, the method comprising: administering a therapeutically effective amount of a compound comprising a multivalent sialyloligosaccharide residue, which is represented by Chemical Formula 1, an optical isomer thereof or a pharmaceutically acceptable salt thereof to an individual.

In addition, the present invention provides a use of a composition comprising a compound comprising a multivalent sialyloligosaccharide residue, which is represented by Chemical Formula 1, an optical isomer thereof, or a pharmaceutically acceptable salt thereof as an active ingredient for preventing, ameliorating, or treating viral diseases.

More specifically, as the composition for preventing, ameliorating, or treating viral infectious diseases, a pharmaceutical composition or animal drug for preventing or treating viral infectious diseases; and a health functional food, animal feed additive, or quasi-drug composition for preventing or ameliorating viral infectious diseases are provided.

Examples of the viral infectious disease include diseases caused by viruses, such as the flu, the common cold, novel flu, pharyngolaryngitis, bronchitis, pneumonia, enteritis, hand-foot-mouth disease, viral meningitis, encephalitis, neurogenic pulmonary edema, conjunctivitis, encephalomyocarditis, myocarditis, hepatitis, poliomyelitis, paralysis, vesicular diseases, herpangina, chronic obstructive pulmonary disease, empyema, otitis media, foot-and-mouth disease, African swine fever, chicken pox, herpes zoster, AIDS, herpes, noroviral enteritis, avian flu, and swine flu.

Examples of the virus include influenza viruses, parainfluenza viruses, adenoviruses, respiratory syncytial virus, rhinoviruses, coronaviruses, and viruses in which two or more of the above viruses are mixed, and are preferably influenza viruses, but are not limited thereto.

The influenza viruses are classified into types A, B, and C, and the type A virus is mainly confirmed to infect people, and is a virus confirmed to infect pigs, other mammals, and various wild birds compared to type B or C, and avian influenza virus, swine influenza virus, influenza A virus subtype H1N1, and the like, which have recently become problems all over the world, belong to this category. More preferably, the influenza virus is influenza virus type A or influenza virus type B.

The influenza virus type A is one or more selected from the group consisting H1N1, H1N2, H2N2, H2N3, H3N1, H3N2, H3N8, H5N1, H5N2, H5N3, H5N8, H5N9, H7N1, H7N2, H7N3, H7N4, H7N7, H7N9, H9N2, and H10N7, and is preferably one or more selected from the group consisting of H1N1, H3N2, H5N1, and H9N2, but is not particularly limited thereto.

The coronavirus is confirmed to infect mammals such as dogs, pigs and cows and birds, and MERS-coronavirus, SARS-coronavirus, coronavirus 229E, coronavirus OC43, coronavirus NL63, canine coronavirus, bovine coronavirus, porcine respiratory coronavirus, porcine epidemic diarrhea virus, avian infectious bronchitis virus, and the like belong to this.

As used herein, the terms “treatment”, “prevention”, and “amelioration” refers to an approach for obtaining beneficial or favorable outcomes, including clinical outcomes. The beneficial or favorable clinical outcomes are not limited to, but may include palliation or amelioration of one or more symptoms or conditions, reduction of the severity of a disease, stabilization of a disease state, prevention of the occurrence of a disease, prevention of the spread of a disease, delay or slowing of disease progression, amelioration or alleviation of a disease state, and remission (whether partial or systemic), whether detectable or undetectable. “Treatment” may also mean prolonging survival as compared to expected survival without treatment.

Further, “treatment” may mean suppressing the progression of a disease and temporarily slowing the progression of a disease, but this may also be accompanied by a permanent arrest of the progression of a disease. As can be understood by those skilled in the art, the outcomes may not be beneficial or favorable if the treatment causes a greater adverse effect on a treated individual than any benefit affected by the treatment while ameliorating a particular disease state.

The pharmaceutical composition according to the present invention may be formulated into a suitable form with a generally used pharmaceutically acceptable carrier. The “pharmaceutically acceptable” means that the pharmaceutical composition is physiologically acceptable and does not typically cause allergic reactions such as gastrointestinal disorders and vertigo or similar reactions when administered to a human. Further, the composition may be used by being formulated in the form of an oral formulation such as a powder, a granule, a pill, a capsule, a suspension, an emulsion, a syrup, and an aerosol, an external preparation, a suppository, and a sterile injection solution, according to a typical method.

Examples of a carrier, an excipient, and a diluent, which may be included in the composition, include lactose, dextrose, sucrose, sorbitol, mannitol, xylitol, erythritol, maltitol, starch, arabic rubber, alginate, gelatin, calcium phosphate, calcium silicate, cellulose, methyl cellulose, microcrystalline cellulose, polyvinylpyrrolidone, water, methyl p-hydroxybenzoate, propyl p-hydroxybenzoate, talc, magnesium stearate, and mineral oil, but are not limited thereto. When the composition is formulated, the composition is prepared using a diluent or excipient, such as a filler, a stabilizer, a binder, a disintegrant, and a surfactant, which are commonly used.

A solid formulation for oral administration includes a tablet, a pill, a powder, a granule, a capsule, and the like, and the solid formulation is prepared by mixing at least one excipient, for example, starch, microcrystalline cellulose, sucrose or lactose, low-substitution-degree hydroxypropyl cellulose, hypromellose, and the like with a compound represented by Chemical Formula 1 of the present invention.

Further, in addition to a simple excipient, lubricants such as magnesium stearate and talc are also used. A liquid formulation for oral administration corresponds to a suspension, a liquid for internal use, an emulsion, a syrup, and the like, and the liquid formulation may include, in addition to water and liquid paraffin which are commonly used simple diluents, various excipients, for example, a wetting agent, a sweetener, an aromatic, a preservative, and the like.

Examples of a formulation for parenteral administration include an aqueous sterile solution, a non-aqueous solvent, a suspension, an emulsion, a freeze-dried preparation, and a suppository. As a non-aqueous solvent and a suspension solvent, it is possible to use propylene glycol, polyethylene glycol, a vegetable oil such as olive oil, an injectable ester such as ethyl oleate, and the like. As a base of the suppository, it is possible to use Witepsol, Macrogol, Tween 61, cacao butter, laurin fat, glycerol, gelatin, and the like. In order to formulate the composition into a formulation for parenteral administration, the compound of Chemical Formula 1 or a pharmaceutically acceptable salt thereof may be sterilized and/or mixed with an adjuvant such as a preservative, a stabilizer, a hydrating agent or an emulsifying agent, a salt and/or a buffer for controlling osmotic pressure, and other therapeutically useful materials in water to be prepared into a solution or a suspension, and the solution or the suspension may be prepared in an ampoule or vial unit administration form.

The compound according to the present invention may be administered to a mammal such as a rat, livestock, and a human via various routes. All modes of administration can be expected, but the compound according to the present invention may be administered, for example, by oral, intraperitoneal, rectal, intravenous, intramuscular, subcutaneous, transdermal, intrapulmonary, intranasal, intrauterine epidural, or intracerebroventricular injection, and the like, or methods of applying, spraying, and inhaling the composition according to the present invention to a diseased site may also be used, and the administration method is not particularly limited.

The therapeutically effective amount or dosage will vary depending on the age, sex, and body weight of a subject to be treated, the particular disease or pathological condition to be treated, the severity of the disease or pathological condition, the administration time, the route of administration, the absorption, distribution and excretion rate of a drug, the types of other drugs used, the judgment of a prescriber, and the like. The determination of the dosage based on these factors is within the capabilities of those skilled in the art, and generally, the dosage is within a range of about 0.01 mg/kg/day to about 2,000 mg/kg/day. A more preferred dosage is 1 mg/kg/day to 500 mg/kg/day. The administration may be carried out once daily, and may be divided into several times. The dosage is not intended to limit the scope of the present invention in any way.

As used herein, the term “individual” refers to any individual member of the animal kingdom, including humans. Examples of mammals include, but are not limited to, any member of the class Mammalia: humans, non-human primates, such as chimpanzees and other apes and monkey species; farm livestock such as cattle, horses, sheep, goats, pigs; animals for pets, such as rabbits, dogs, cats. In some exemplary embodiments of the method and composition provided by the present invention, the mammal is a human or a non-human mammal.

The health functional food refers to a food manufactured or processed using raw materials and components having useful functionality, and examples thereof include all of health supplement foods, functional foods, nutrients, supplements, and the like.

The health functional food may include a compound comprising a multivalent sialyloligosaccharide residue and a sitologically acceptable food supplement additive, and the compound may be added in an amount of preferably 0.001 wt % to 50 wt %, more preferably 0.001 wt % to 30 wt %, and most preferably 0.001 wt % to 10 wt % based on the total weight of the entire health functional food.

The health functional food of the present invention includes a form such as a tablet, a capsule, a pill or a liquid, and examples of a food to which the compound of the present invention can be added include various foods, beverages, gums, teas and vitamin complexes, and the like.

The animal feed additive can be used as an additive in various poultry feeds including those for poultry farming, and in this case, the poultry feed is sufficient as long as it is a feed generally used by livestock farmers.

For the animal feed additive, the compound of the present invention may be added to the composition for animal feed in an amount of 0.001 wt % to 30 wt %, preferably 0.001 wt % to 10 wt %, and most preferably 0.001 wt % to 5 wt %.

The animal feed additive of the present invention serves as an auxiliary component of a composition for animal feed, and after various adjuvants such as amino acids, inorganic salts, vitamins, antibiotic materials, antibacterial materials, antioxidants, antifungal enzymes, and live microbial preparations are mixed with grains, such as pulverized or crushed wheat, oats, barley, corn, and rice; vegetable protein feeds, such as those including rapes, soybeans, and sunflowers as main components; animal protein feeds, such as blood meal, meat meal, bone meal, and fish meal; dry components including sugar and dairy products, such as various powdered milk and whey powder, and dry additives, a liquid component may be used together with a material such as a nutritional supplement, a digestion and absorption enhancer, a growth promoter, and a disease prevention agent may be used together, in addition to a component that becomes liquid after heating, that is, a main component such as lipids such as animal fat and vegetable fat arbitrarily liquefied by heating. When the animal feed additive is administered separately from the animal feed, as is well known in the art, the administration form of a dry powder or an extract may be prepared in an immediate release or sustained release dosage form in combination with a non-toxic pharmaceutically acceptable edible carrier. The edible carrier may be a solid or a liquid, such as corn starch, lactose, sucrose, bean flakes, peanut oil, olive oil, sesame oil and propylene glycol. When a solid carrier is used, the dosage form of the dry powder or the extract may be a tablet, a capsule, a powder, a troche, or a top dressing in a sugar-containing tablet or microdispersed form. When a liquid carrier is used, the liquid carrier may be in the administration form of a soft gelatin capsule, or a syrup or liquid suspension, an emulsion or a solution. In addition, the administration form may contain an adjuvant such as a preservative, a stabilizer, a wetting agent or an emulsifier, and a solution accelerator.

The quasi-drug is an article that is a textile, a rubber product or a similar product used for the purpose of treating, alleviating, disposing of, or preventing human or animal diseases, has a weak effect on the human body or does not act directly on the human body, is not an instrument or a machine, and is a similar product, and corresponds to one among the preparations used for sterilization, insecticidal and similar uses for prevention of infectious diseases, and refers to an article except for those which are not an instrument, a machine, or a device among articles used for the purpose of treating, alleviating, disposing of, or preventing human or animal diseases, and is not an instrument, a machine, or a device among articles used for the purpose of pharmacologically affecting the structure and function of humans or animals.

The type or dosage form of the quasi-drug composition of the present invention is not particularly limited, and the quasi-drug composition of the present invention exhibits antiviral activity and may be formulated in various forms of quasi-drugs known in the art. The formulated quasi-drug includes a disinfectant cleansing agent, a shower foam, a mouthwash, a wet tissue, a detergent soap, a hand wash, an ointment, a cream, a lotion, a spray, a humidifier filler, a mask, a patch, a filter filler, or the like, and includes all quasi-drugs in a typical sense.

When the compound of the present invention is added to a quasi-drug composition for the purpose of preventing or ameliorating viral infectious diseases, the compound may be added as it is or used together with other quasi-drugs or quasi-drug components, and may be used appropriately by a typical method. The amount of active ingredient mixed may be suitably determined depending on the intended use and may include, for example, a typical adjuvant such as a thickener, a stabilizer, a solubilizer, a vitamin, a pigment and a fragrance, and a carrier, and the like. Hereinafter, preferred Examples and Experimental Examples of the present invention will be described in detail with reference to the accompanying drawings. However, these Examples and Experimental Examples are provided only for exemplifying the present invention, and it is not to be interpreted that the scope of the present invention is limited by these Examples and Experimental Examples.

### Example 1. Synthesis of Compounds 1 and 2

Compound 1 (Hex-AH-3′SL) and Compound 2 (Hex-AH-6′SL) of the present invention were synthesized with reference to the following Reaction Schemes 1 and 2.

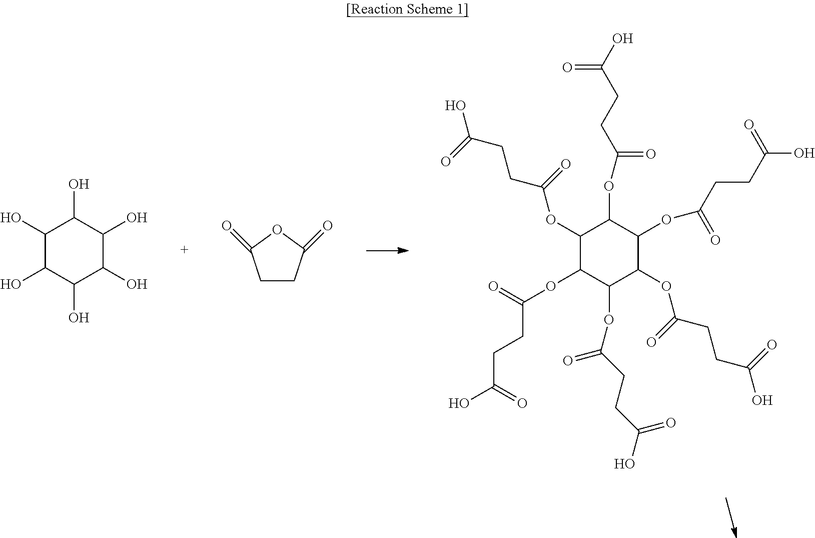 [SMILES: OC1C(O)C(O)C(O)C(O)C1O ; O=C1CCC(=O)O1 ; O=C(O)CCC(=O)OC1C(OC(=O)CCC(=O)O)C(OC(=O)CCC(=O)O)C(OC(=O)CCC(=O)O)C(OC(=O)CCC(=O)O)C1OC(=O)CCC(=O)O]

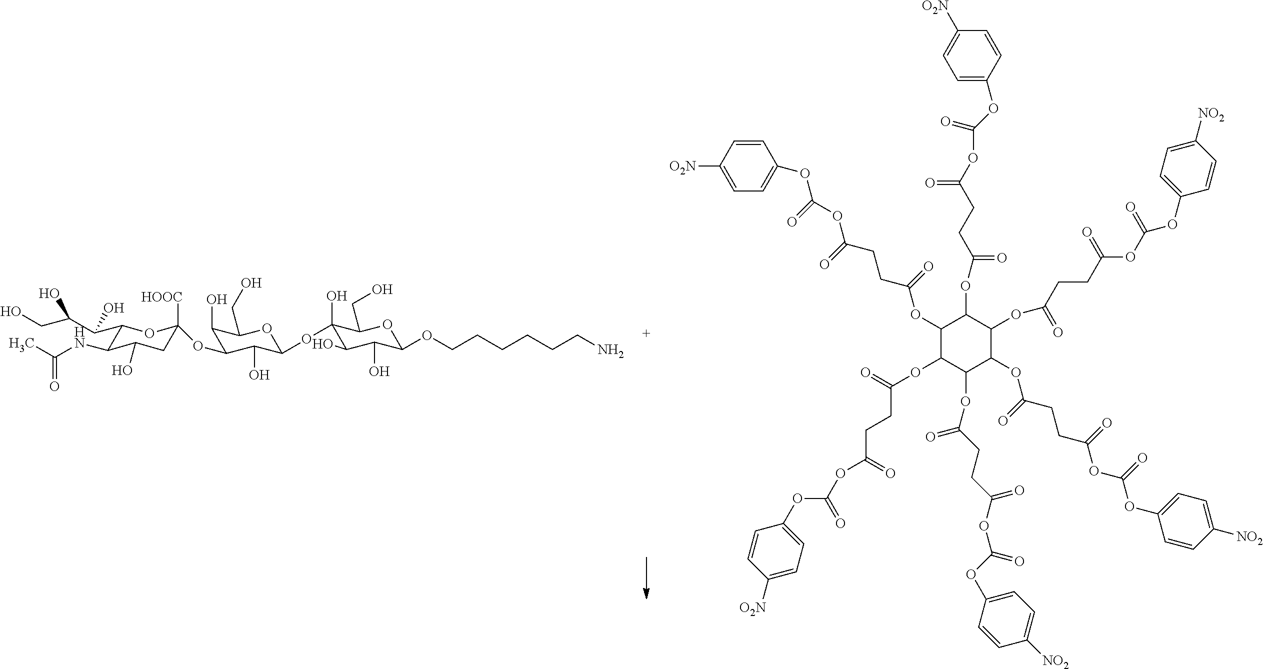 [SMILES: CC(=O)NC1C(O)CC(OC2C(O)C(CO)OC(OC3(O)C(CO)OC(OCCCCCCN)C(O)C3O)C2O)(C(=O)O)OC1[C@H](O)[C@H](O)CO ; O=C(CCC(=O)OC1C(OC(=O)CCC(=O)OC(=O)Oc2ccc([N+](=O)[O-])cc2)C(OC(=O)CCC(=O)OC(=O)Oc2ccc([N+](=O)[O-])cc2)C(OC(=O)CCC(=O)OC(=O)Oc2ccc([N+](=O)[O-])cc2)C(OC(=O)CCC(=O)OC(=O)Oc2ccc([N+](=O)[O-])cc2)C1OC(=O)CCC(=O)OC(=O)Oc1ccc([N+](=O)[O-])cc1)OC(=O)Oc1ccc([N+](=O)[O-])cc1]

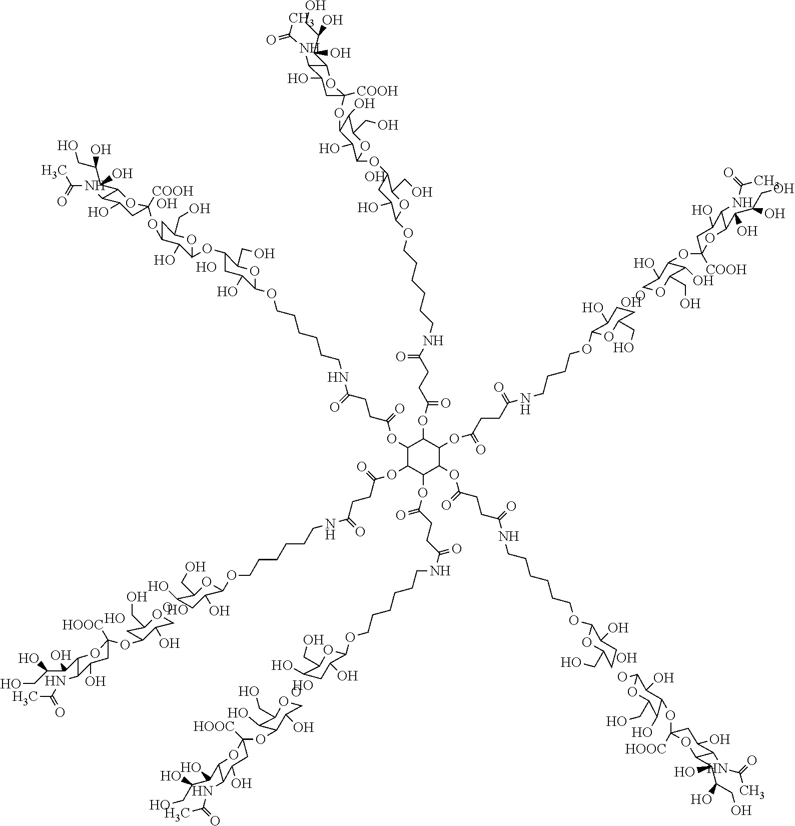

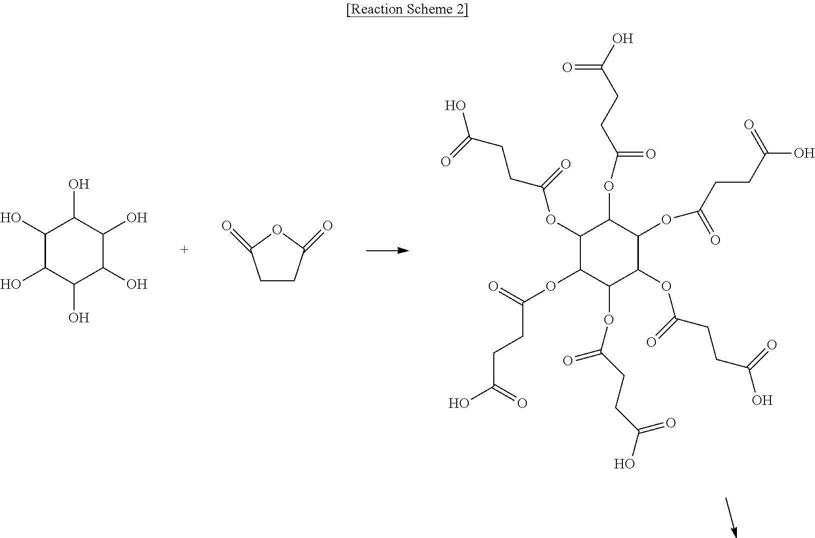 [SMILES: OC1C(O)C(O)C(O)C(O)C1O ; O=C1CCC(=O)O1 ; O=C(O)CCC(=O)OC1C(OC(=O)CCC(=O)O)C(OC(=O)CCC(=O)O)C(OC(=O)CCC(=O)O)C(OC(=O)CCC(=O)O)C1OC(=O)CCC(=O)O]

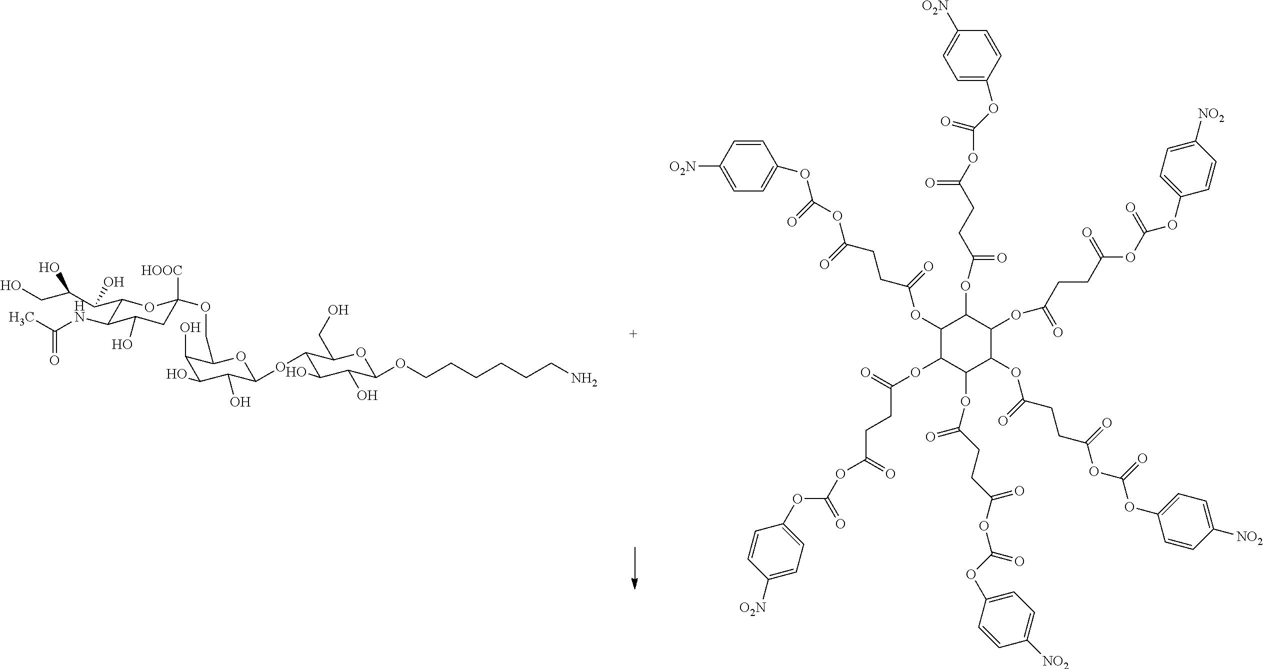 [SMILES: CC(=O)NC1C(O)CC(OCC2OC(OC3C(CO)OC(OCCCCCCN)C(O)C3O)C(O)C(O)C2O)(C(=O)O)OC1[C@H](O)[C@H](O)CO ; O=C(CCC(=O)OC1C(OC(=O)CCC(=O)OC(=O)Oc2ccc([N+](=O)[O-])cc2)C(OC(=O)CCC(=O)OC(=O)Oc2ccc([N+](=O)[O-])cc2)C(OC(=O)CCC(=O)OC(=O)Oc2ccc([N+](=O)[O-])cc2)C(OC(=O)CCC(=O)OC(=O)Oc2ccc([N+](=O)[O-])cc2)C1OC(=O)CCC(=O)OC(=O)Oc1ccc([N+](=O)[O-])cc1)OC(=O)Oc1ccc([N+](=O)[O-])cc1]

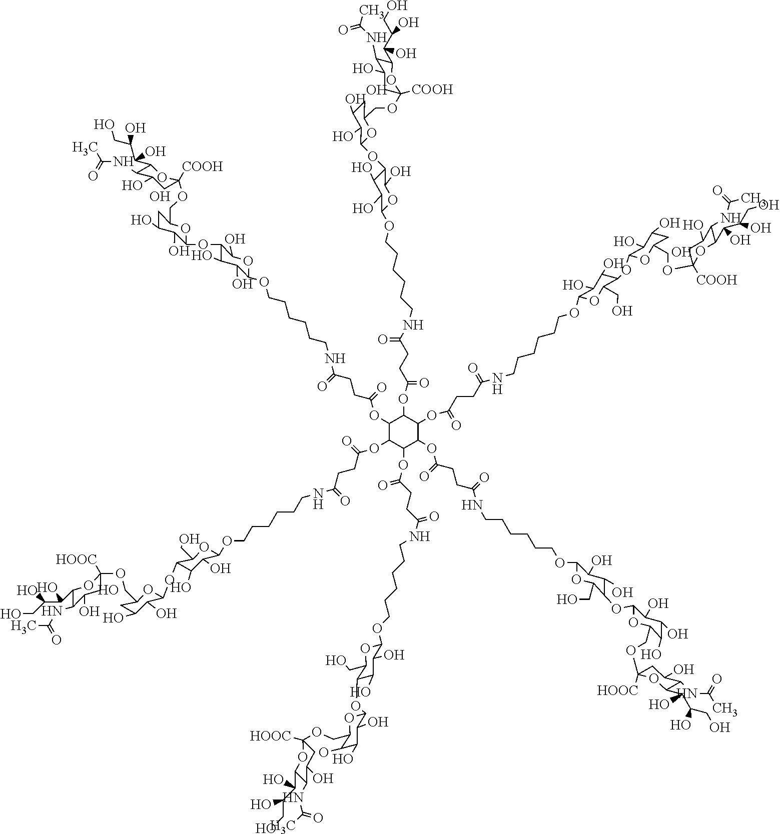

### Example 1-1. Synthesis of Succinyl Inositol

After 200 ml of pyridine, 15.5 ml of TEA, and 8.1 g of DMAP were put into 10 g of inositol and the resulting mixture was stirred, 6.1 g of succinic anhydride was added thereto, and the resulting mixture was reacted. The reaction product was purified with silica gel to obtain succinyl inositol.

### Example 1-2. Synthesis of Inositol Succinyl Active Ester

After 200 ml of pyridine and 1.88 g of DMAP were put into the succinyl inositol of Example 1-1 and the resulting mixture was stirred, 20.6 g of 4-nitrophenyl chloroformate was added thereto, and the resulting mixture was reacted. After completion of the reaction, the reaction product was concentrated, and then purified with silica gel to obtain inositol succinyl active ester.

### Example 1-3. Synthesis of Compounds 1 and 2

After 50 ml of pyridine and 1.2 ml of TEA were put into 1.36 g of aminohexyl 3′-sialyl lactose (aminohexyl 3′-SL) or aminohexyl 6′-sialyl lactose (aminohexyl 6′-SL) and the resulting mixture was stirred, 0.5 g of the inositol succinyl active ester of Example 1-2 was dissolved in 50 ml of pyridine, and the resulting solution was slowly added dropwise thereto. After completion of the reaction, the reaction product was concentrated, and then purified with size exclusion chromatography to obtain Compound 1 (Hex-AH-3′SL) or Compound 2 (Hex-AH-6′SL).

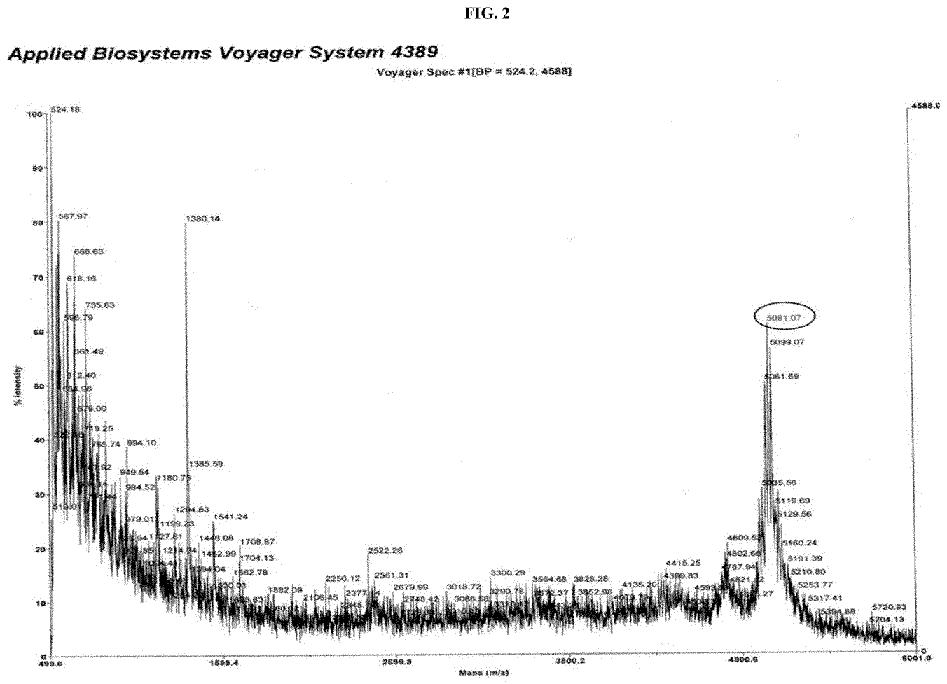

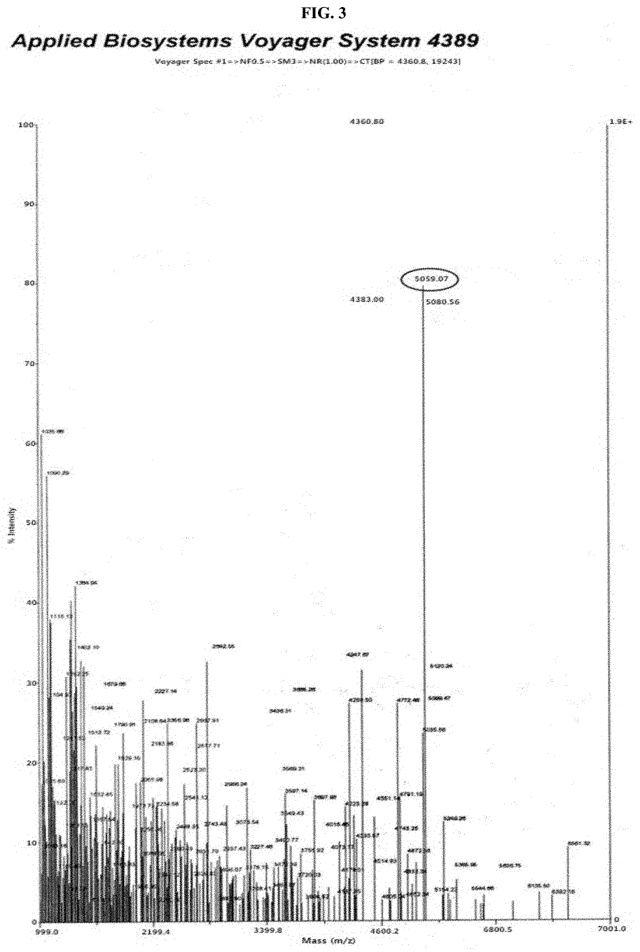

Compounds 1 and 2 obtained from the above process are shown in FIGS. 2 and 3 by confirming the molecular weight of the final material by MALDI-TOF. When looking at FIGS. 2 and 3, a spectral analysis value for Compound 1 is shown to be 5081.07 g/mol which is interpreted as [M+3Na−3H₂O−2H]⁺, and a spectral value for Compound 2 is shown to be 5069.07 g/mol which is interpreted as [M+2Na−3H₂O−H]⁺.

### Example 2. Synthesis of Compounds 3 and 4

Compound 3 (Tetra-AH-3′SL) and Compound 4 (Tetra-AH-6′SL) were synthesized with reference to the following Reaction Schemes 3 and 4.

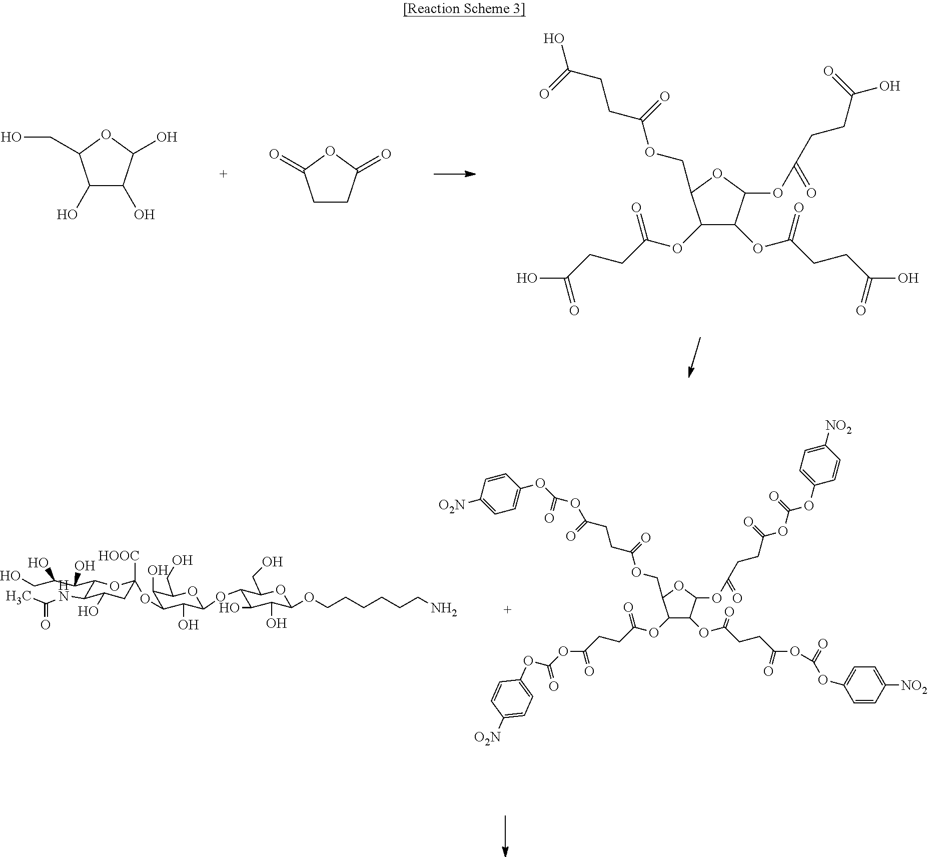

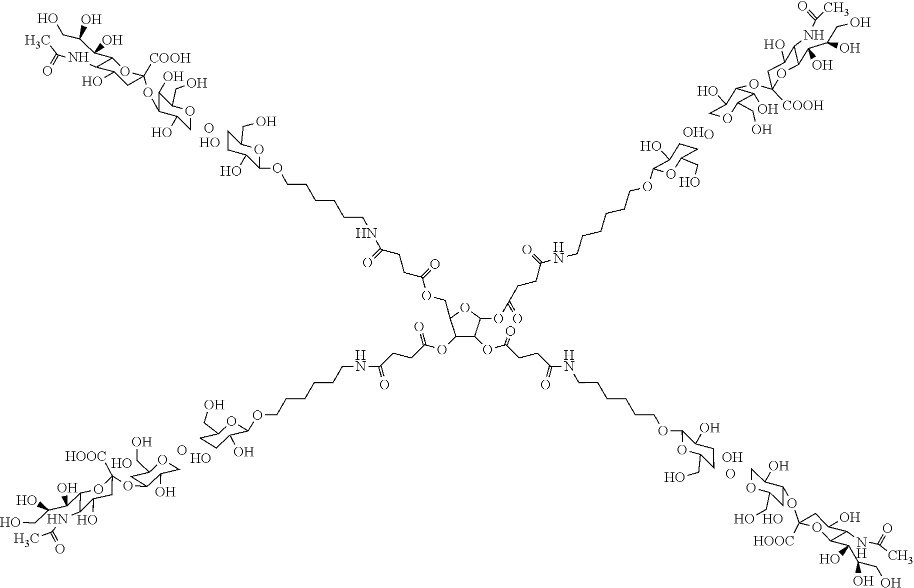

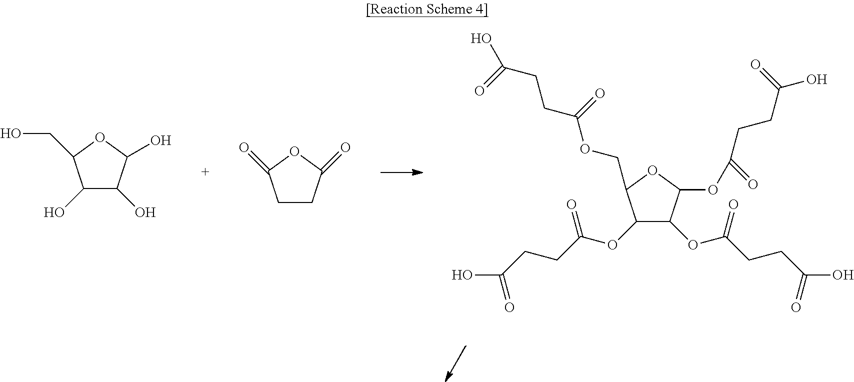 [SMILES: OCC1OC(O)C(O)C1O ; O=C1CCC(=O)O1 ; O=C(O)CCC(=O)OCC1OC(OC(=O)CCC(=O)O)C(OC(=O)CCC(=O)O)C1OC(=O)CCC(=O)O]

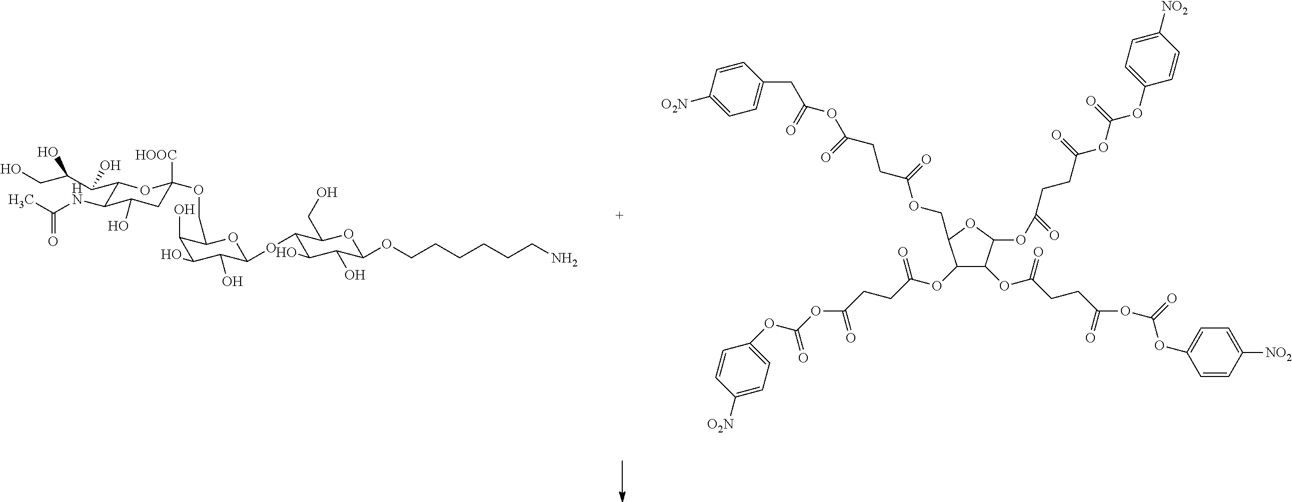 [SMILES: CC(=O)NC1C(O)CC(OCC2OC(OC3C(CO)OC(OCCCCCCN)C(O)C3O)C(O)C(O)C2O)(C(=O)O)OC1[C@H](O)[C@H](O)CO ; O=C(CCC(=O)OC(=O)Cc1ccc([N+](=O)[O-])cc1)OCC1OC(OC(=O)CCC(=O)OC(=O)Oc2ccc([N+](=O)[O-])cc2)C(OC(=O)CCC(=O)OC(=O)Oc2ccc([N+](=O)[O-])cc2)C1OC(=O)CCC(=O)OC(=O)Oc1ccc([N+](=O)[O-])cc1]

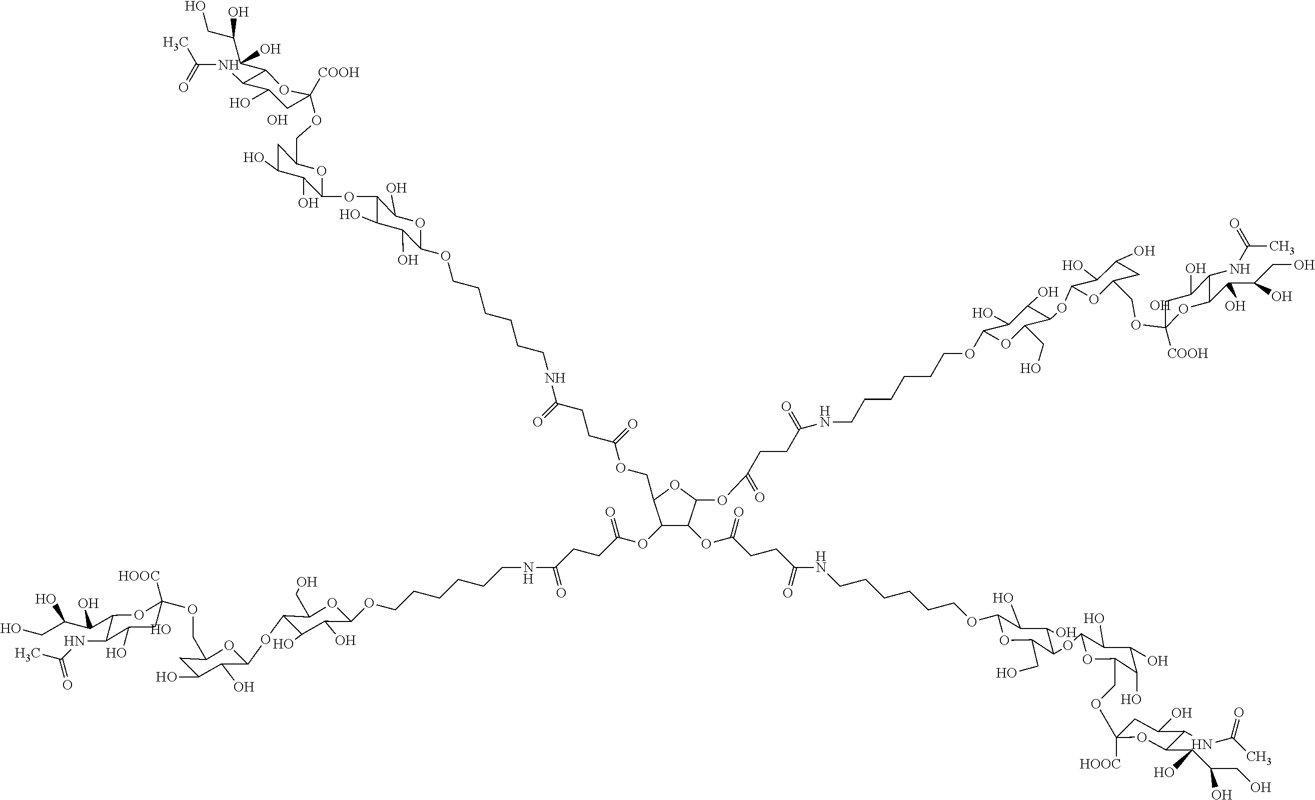

### Example 2-1. Synthesis of Succinyl Ribose

After 30 ml of pyridine, 5 ml of TEA, and 1.95 g of DMAP were put into 3 g of ribose and the resulting mixture was stirred, 2.2 g of succinic anhydride was added thereto, and the resulting mixture was reacted. The reaction product was purified with silica gel to obtain succinyl ribose.

### Example 2-2. Synthesis of Ribose Succinyl Active Ester

After 50 ml of pyridine and 3.9 g of DMAP were put into the succinyl ribose of Example 2-1 and the resulting mixture was stirred, 6.44 g of 4-nitrophenyl chloroformate was added thereto, and the resulting mixture was stirred. After completion of the reaction, the reaction product was concentrated, and then purified with silica gel to obtain ribose succinyl active ester.

### Example 2-3. Synthesis of Compounds 3 and 4

After 8.5 ml of pyridine and 0.11 ml of TEA were put into 0.22 g of aminohexyl 3′-sialyl lactose (aminohexyl 3′-SL) or aminohexyl 6′-sialyl lactose (aminohexyl 6′-SL) and the resulting mixture was stirred, 0.05 g of the ribose succinyl active ester of Example 2-2 was dissolved in 4.3 ml of pyridine, and the resulting solution was slowly added dropwise thereto. After completion of the reaction, the reaction product was concentrated, and then purified with size exclusion chromatography to obtain Compound 3 (Tetra-AH-3′SL) or Compound 4 (Tetra-AH-6′SL).

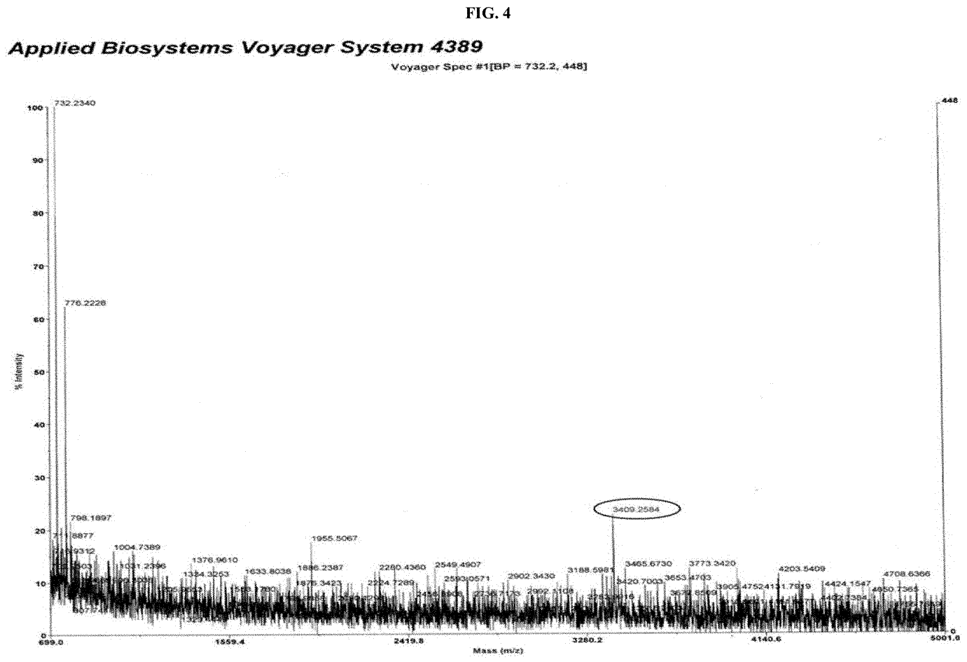

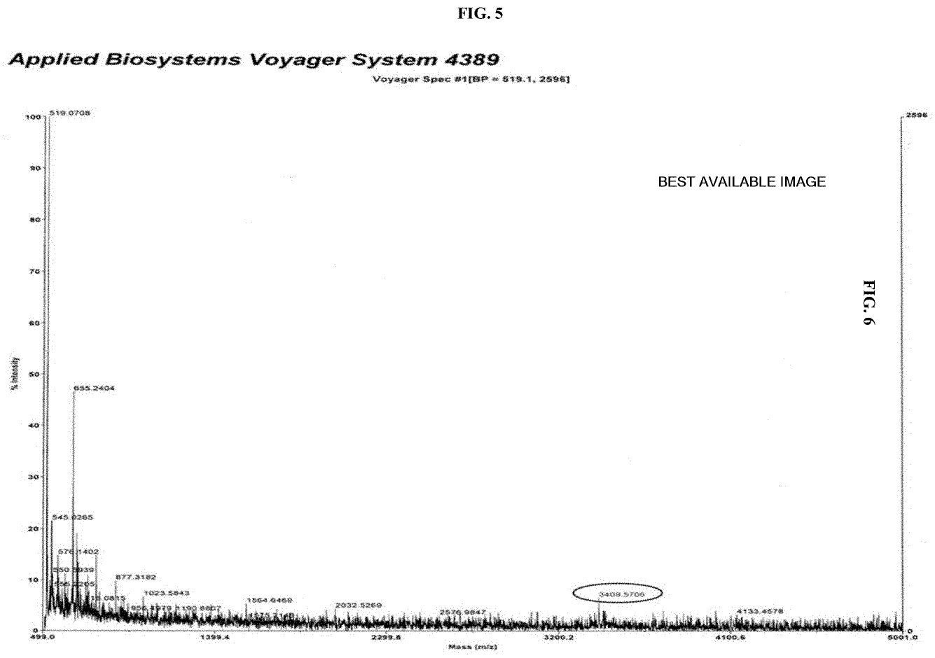

Compounds 3 and 4 obtained from the above process are shown in FIGS. 4 and 5 by confirming the molecular weight of the final material by MALDI-TOF. When looking at FIGS. 4 and 5, Compound 3 shows a molecular weight of 3409.26 g/mol, and Compound 4 shows a molecular weight of 3409.57 g/mol.

### Example 3. Synthesis of Compounds 5 and 6

Compound 5 (Hexa-carboxyl-3′SL) and Compound 6 (Hexa-carboxyl-6′SL) were synthesized with reference to the following Reaction Schemes 5 and 6.

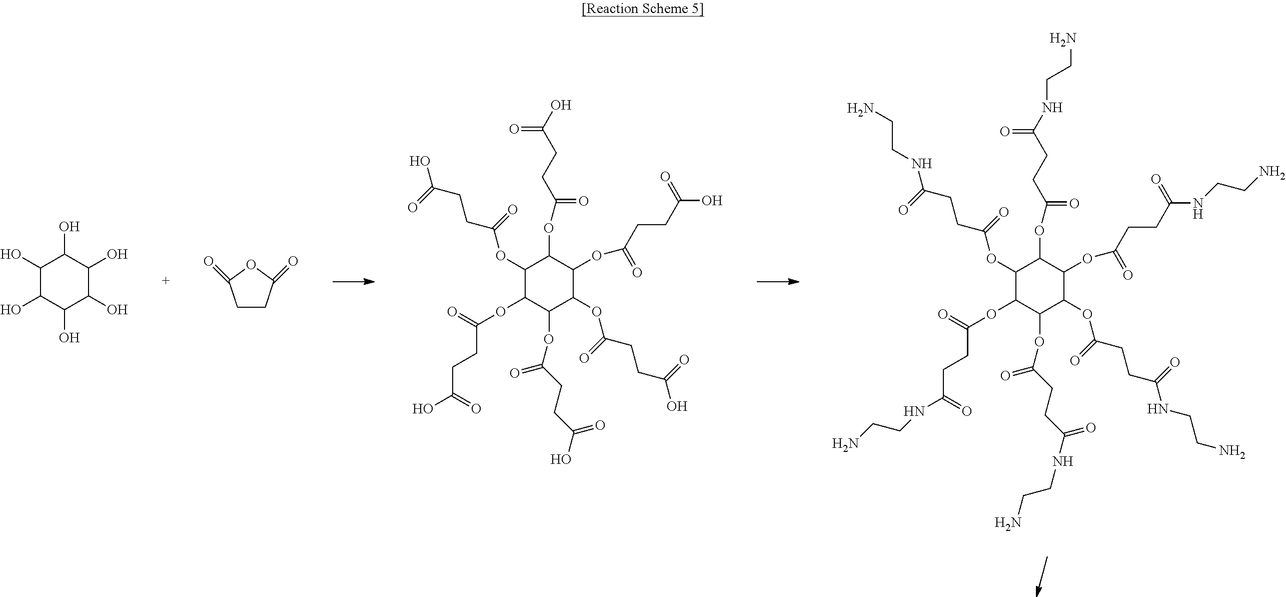 [SMILES: OC1C(O)C(O)C(O)C(O)C1O ; O=C1CCC(=O)O1 ; O=C(O)CCC(=O)OC1C(OC(=O)CCC(=O)O)C(OC(=O)CCC(=O)O)C(OC(=O)CCC(=O)O)C(OC(=O)CCC(=O)O)C1OC(=O)CCC(=O)O ; NCCNC(=O)CCC(=O)OC1C(OC(=O)CCC(=O)NCCN)C(OC(=O)CCC(=O)NCCN)C(OC(=O)CCC(=O)NCCN)C(OC(=O)CCC(=O)NCCN)C1OC(=O)CCC(=O)NCCN]

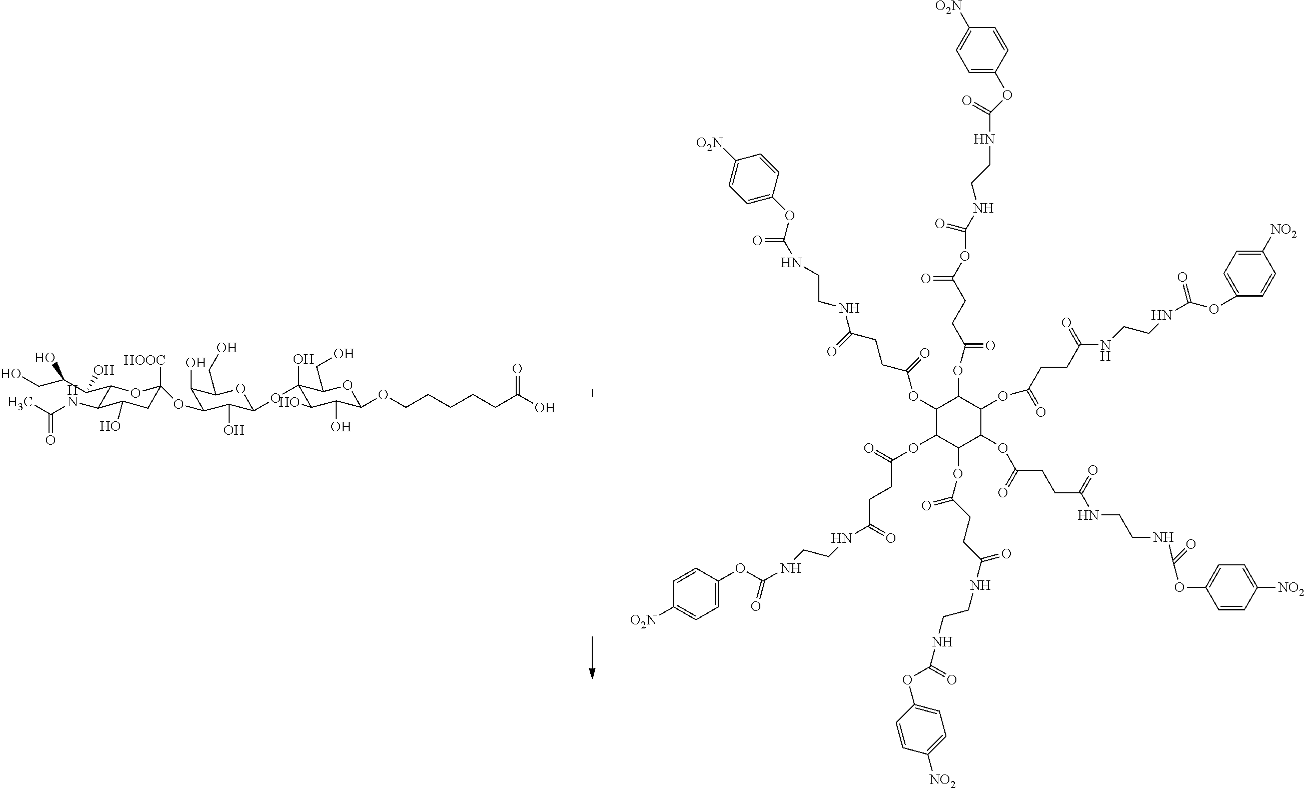 [SMILES: CC(=O)NC1C(O)CC(OC2C(O)C(CO)OC(OC3(O)C(CO)OC(OCCCCCC(=O)O)C(O)C3O)C2O)(C(=O)O)OC1[C@H](O)[C@H](O)CO ; O=C(CCC(=O)OC1C(OC(=O)CCC(=O)NCCNC(=O)Oc2ccc([N+](=O)[O-])cc2)C(OC(=O)CCC(=O)NCCNC(=O)Oc2ccc([N+](=O)[O-])cc2)C(OC(=O)CCC(=O)OC(=O)NCCNC(=O)Oc2ccc([N+](=O)[O-])cc2)C(OC(=O)CCC(=O)NCCNC(=O)Oc2ccc([N+](=O)[O-])cc2)C1OC(=O)CCC(=O)NCCNC(=O)Oc1ccc([N+](=O)[O-])cc1)NCCNC(=O)Oc1ccc([N+](=O)[O-])cc1]

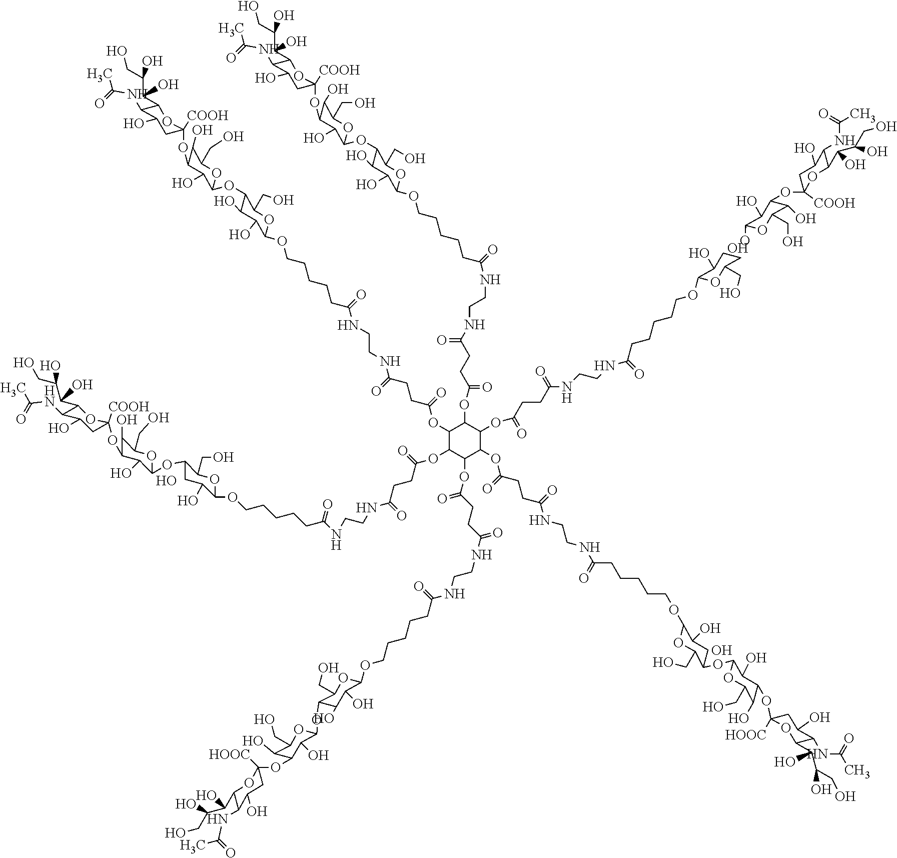

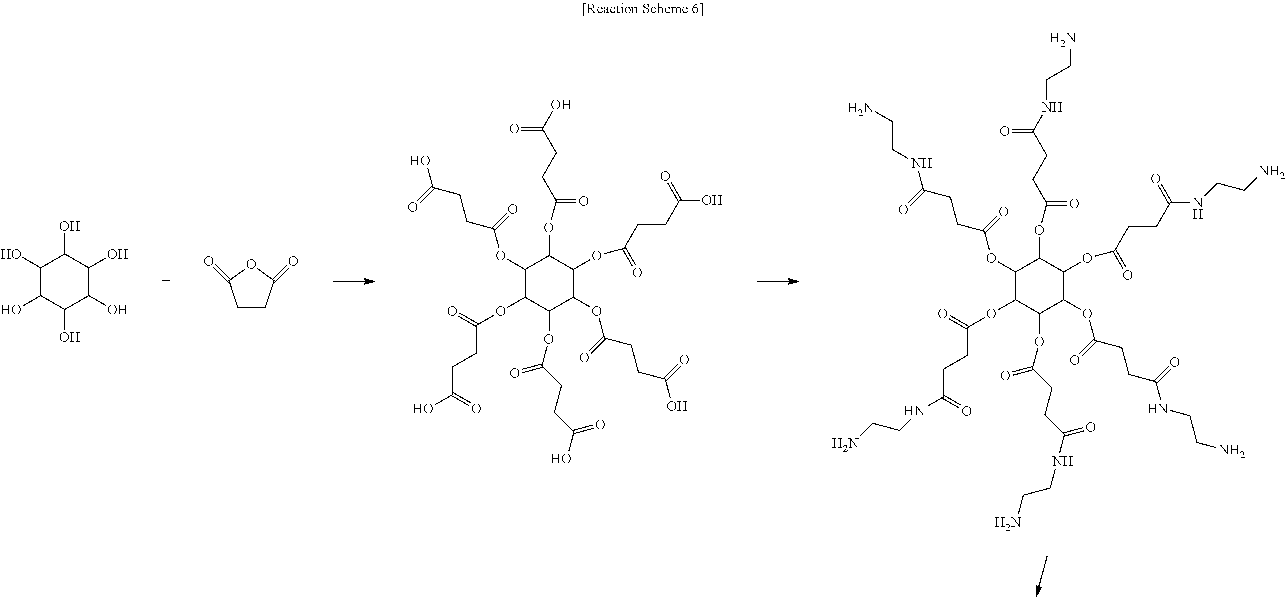 [SMILES: OC1C(O)C(O)C(O)C(O)C1O ; O=C1CCC(=O)O1 ; O=C(O)CCC(=O)OC1C(OC(=O)CCC(=O)O)C(OC(=O)CCC(=O)O)C(OC(=O)CCC(=O)O)C(OC(=O)CCC(=O)O)C1OC(=O)CCC(=O)O ; NCCNC(=O)CCC(=O)OC1C(OC(=O)CCC(=O)NCCN)C(OC(=O)CCC(=O)NCCN)C(OC(=O)CCC(=O)NCCN)C(OC(=O)CCC(=O)NCCN)C1OC(=O)CCC(=O)NCCN]

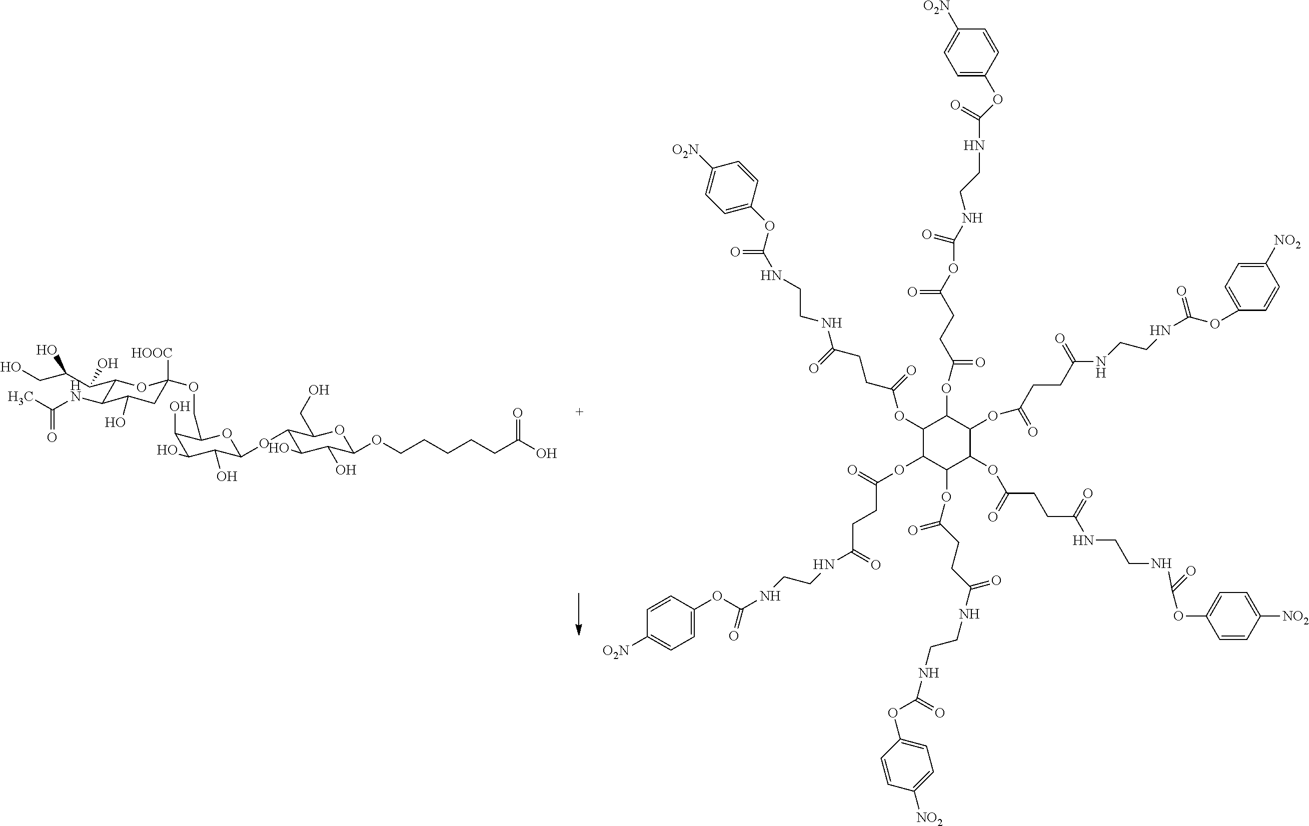 [SMILES: CC(=O)NC1C(O)CC(OCC2OC(OC3C(CO)OC(OCCCCCC(=O)O)C(O)C3O)C(O)C(O)C2O)(C(=O)O)OC1[C@H](O)[C@H](O)CO ; O=C(CCC(=O)OC1C(OC(=O)CCC(=O)NCCNC(=O)Oc2ccc([N+](=O)[O-])cc2)C(OC(=O)CCC(=O)NCCNC(=O)Oc2ccc([N+](=O)[O-])cc2)C(OC(=O)CCC(=O)OC(=O)NCCNC(=O)Oc2ccc([N+](=O)[O-])cc2)C(OC(=O)CCC(=O)NCCNC(=O)Oc2ccc([N+](=O)[O-])cc2)C1OC(=O)CCC(=O)NCCNC(=O)Oc1ccc([N+](=O)[O-])cc1)NCCNC(=O)Oc1ccc([N+](=O)[O-])cc1]

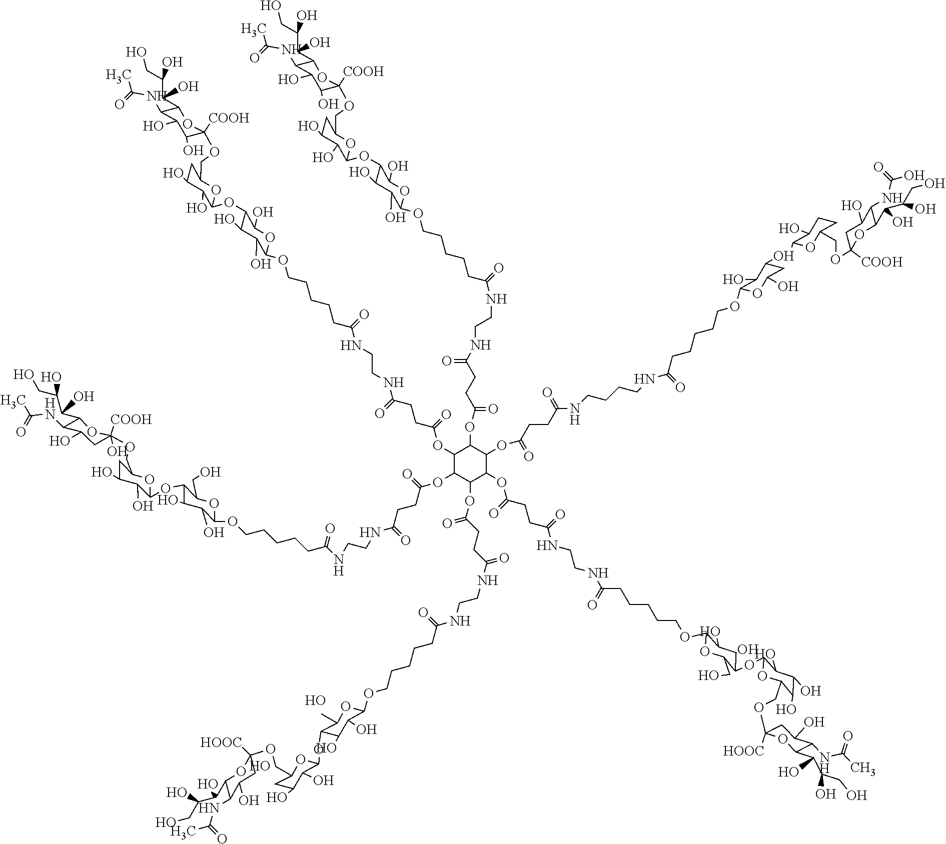

### Example 3-1. Synthesis of Succinyl Inositol

After 200 ml of pyridine, 15.5 ml of TEA, and 8.1 g of DMAP were put into 10 g of inositol and the resulting mixture was stirred, 6.1 g of succinic anhydride was added thereto, and the resulting mixture was reacted. The reaction product was purified with silica gel to obtain succinyl inositol.

### Example 3-2. Synthesis of Inositol Succinyl Diamine

After 200 ml of DMF, 10.3 g of DMAP, and 19 g of DCC were put into the succinyl inositol of Example 3-1 and the resulting mixture was stirred, 6.18 g of ethylene diamine was added thereto, and the resulting mixture was reacted. After completion of the reaction, the reaction product was concentrated, and then purified with silica gel to obtain inositol succinyl diamine.

### Example 3-3. Synthesis of Inositol Succinyl Diamine Active Ester

After 100 ml of pyridine and 0.94 g of DMAP were put into the inositol succinyl diamine of Example 3-2 and the resulting mixture was stirred, 10.3 g of 4-nitrophenylchloroformate was added thereto, and the resulting mixture was reacted. After completion of the reaction, the reaction product was concentrated, and then purified with silica gel to obtain inositol succinyl active ester.

### Example 3-4. Synthesis of Compounds 5 and 6

After 50 ml of pyridine and 12 ml of TEA were put into 2 g of carboxyl 3′-SL or carboxyl 6′-SL and the resulting mixture was stirred, 0.3 g of the inositol succinyl active ester of Example 3-3 was dissolved in 15 ml of pyridine, and the resulting solution was slowly added dropwise thereto. After completion of the reaction, the reaction product was concentrated, and then purified with size exclusion chromatography to obtain Compound 5 and Compound 6.

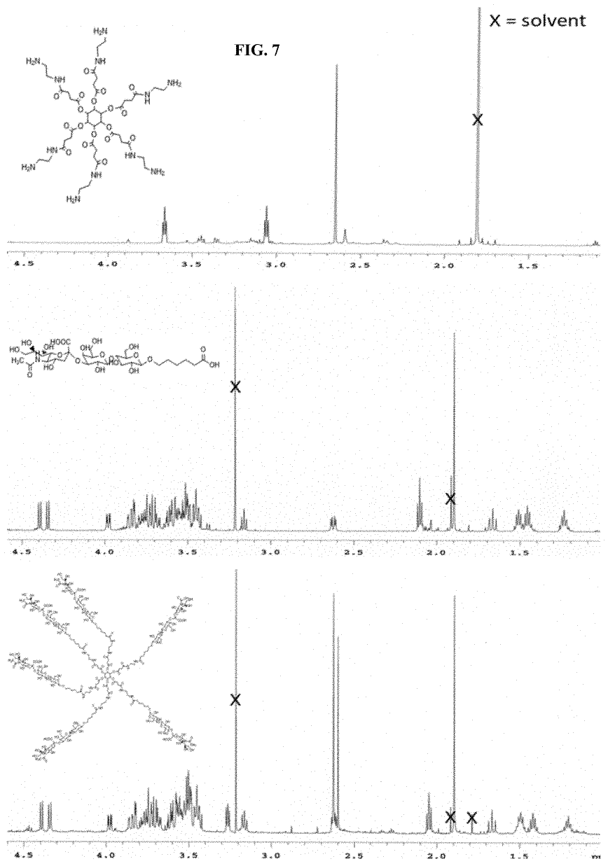

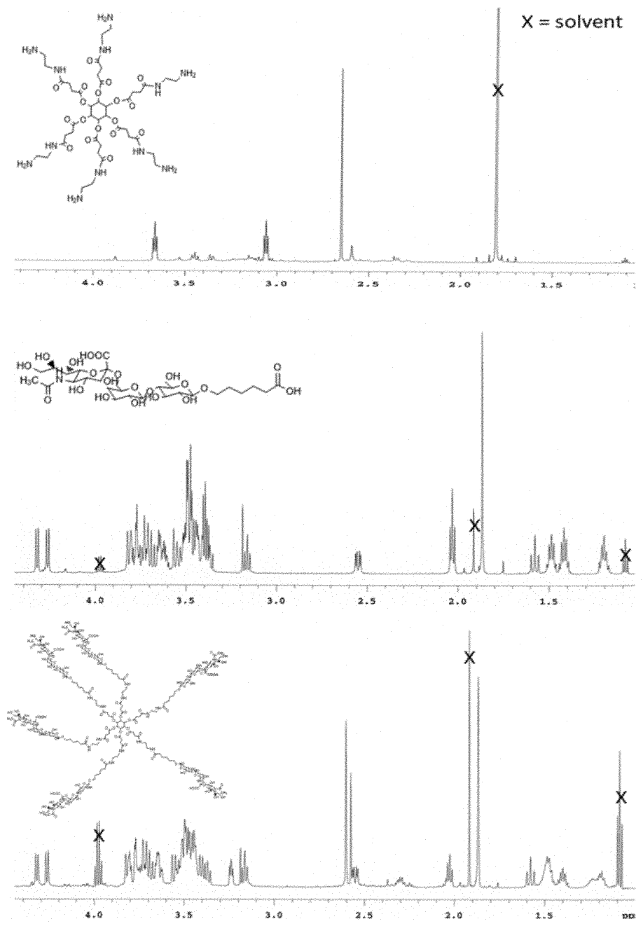

Compound 5 and Compound 6 obtained from the process are shown in FIGS. 6 and 7 by confirming the structure of each compound by NMR.

### Example 4. Confirmation of Antiviral Activity

### Example 4-1. Hemagglutination Inhibition Activity Test

A compound comprising a multivalent sialyloligosaccharide residue was tested for hemagglutination inhibition activity of an influenza virus using chicken red blood cells (cRBCs). After Compounds 1 to 6 (500 mM, 25 μl each) and Comparative Compounds 1 and 2 (500 mM, 25 μl each) were mixed with H1N1 (PR8), H3N2 (swine), and H9N2 (avian) in hemagglutination units (HAU), 25 μl of 1% cRBCs were each added thereto, the resulting was reacted for 90 minutes, and the degree of hemagglutination inhibition is shown in Table 1.

TABLE 1

| | Hemagglutination inhibition concentration (mM) | | | |
|---|---|---|---|---|
| | Conditions | | | |
| | Influenza Type A | | | |
| | H1N1 | H3N2 | H9N2 | Influenza |
| | (PR8) | (swine) | (avian) | Type B |
| Compound 1 | 0.0026 | 2.0 | 0.33 | 0.0043 |
| (Hex-AH- | | | | |
| 3′ SL) | | | | |
| Compound 2 | 0.0015 | 0.65 | 1.3 | 0.0011 |
| (Hex-AH- | | | | |
| 6′ SL) | | | | |
| Compound 3 | 0.85 | 1.0 | 0.54 | 0.22 |
| (Tetra-AH- | | | | |
| 3′ SL) | | | | |
| Compound 4 | 0.45 | 0.85 | 2.0 | 0.15 |
| (Tetra-AH- | | | | |
| 6′ SL) | | | | |
| Compound 5 | 0.005 | 0.018 | 0.21 | 0.108 |
| (Hex-Carboxyl- | | | | |
| 6′ SL) | | | | |
| Compound 6 | 0.05 | 0.009 | 1.11 | 0.031 |
| (Hex-Carboxy- | | | | |
| 3′ SL) | | | | |
| Comparative | 25 | 20 | 18 | 10 |
| Compound 1 | | | | |
| (3′ SL) | | | | |
| Comparative | 12.5 | 4.5 | >100 | 4.5 |
| Compound 2 | | | | |
| (6′ SL) | | | | |

When looking at Table 1, Compounds 1 to 6 show remarkably superior antiviral activity against influenza viruses types A and B compared to Comparative Compounds 1 and 2. Therefore, it could be seen that since the multivalent sialyl lactose comprising a sugar having a cycloalkyl structure of Compounds 1 to 6 of the present invention as a core structure has a high binding force to a virus and excellent hemagglutination inhibition activity against a virus, the multivalent sialyl lactose could be usefully used as a composition for preventing or treating viral infectious diseases.

### Example 4-2. Virus Neutralization Test

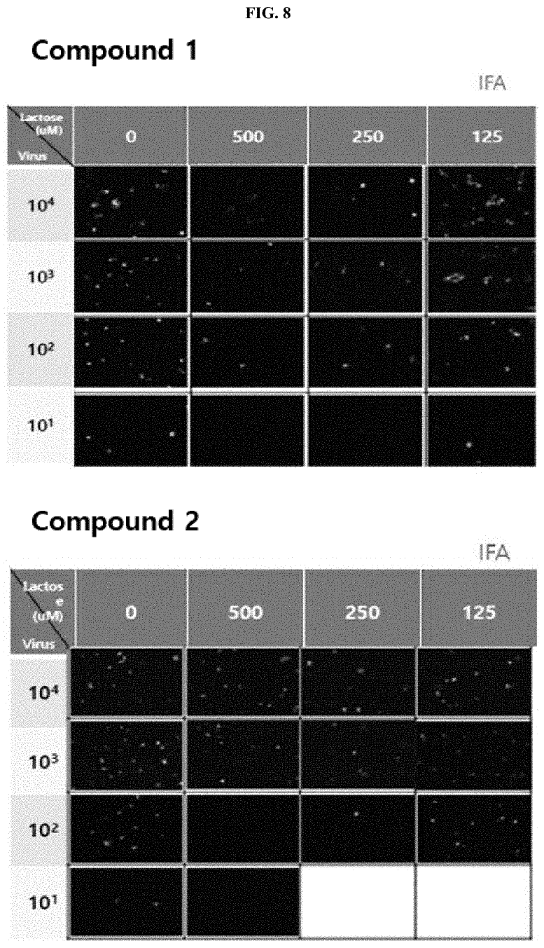

After 10-fold serial dilution of 10,000 EID₅₀/ml of H1N1 (PR8) virus, 50 μl of each viral titer and 50 μl of Compound 1 or 2 at each concentration were mixed, and then reacted at 37° C. for 90 minutes. After 90 minutes, the reaction solution was inoculated into Madin Darby Canine kidney (MDCK) cells and the cells were cultured in a 5% CO2 incubator for 72 to 96 hours. Then, after the supernatant of the culture solution was removed, the infected cells were fixed with an acetone:methanol (1:1) solution, and an anti-influenza virus mouse monoclonal antibody was added thereto. Next, after an anti-mouse FITC conjugate was reacted, the reaction product was observed under a fluorescence microscope, and the observed image is shown in FIG. 8.

Referring to FIG. 8, Compound 1 showed a viral neutralization activity of 10⁵ EID₅₀/ml at 250 to 500 μM, and Compound 2 showed a viral neutralization activity of 10² EID₅₀/ml at 500 μM. Therefore, it could be seen that the compound of the present invention is a composition excellent in antiviral activity.

### Preparation Example 1. Preparation of Tablets

20 g of Compound 1 (Hex-AH-3′SL) of the present invention was mixed with 175.9 g of lactose, 180 g of potato starch, and 32 g of colloidal silicic acid. A 10% gelatin solution was added to this mixture, and then the resulting mixture was pulverized and passed through a 14-mesh sieve. The resultant was dried, and a mixture obtained by adding 160 g of potato starch, 50 g of talc, and 5 g of magnesium stearate thereto was prepared into tablets.

### Preparation Example 2. Preparation of Capsules

After 100 mg of Compound 1 (Hex-AH-3′SL) of the present invention, 100 mg of corn starch, 100 mg of lactose, and 2 mg of magnesium stearate were mixed, capsules were prepared by mixing the aforementioned components according to a typical capsule preparation method and filling the mixture into gelatin capsules.

### Preparation Example 3. Preparation of Injection

1 g of Compound 1 (Hex-AH-3′SL) of the present invention, 0.6 g of sodium chloride, and 0.1 g of ascorbic acid were dissolved in distilled water to make 100 ml. The solution was put into a bottle and sterilized by being heated at 20° C. for 30 minutes.

### Preparation Example 4. Preparation of Health Functional Food

20 g of Compound 1 (Hex-AH-3′SL) of the present invention, a suitable amount of a vitamin mixture, 70 μg of vitamin A acetate, 1.0 mg of vitamin E, 0.13 mg of vitamin B1, 0.15 mg of vitamin B2, 0.5 mg of vitamin B6, 0.2 μg of vitamin B12, 10 mg of vitamin C, 10 μg of biotin, 1.7 mg of nicotinic acid amide, 50 μg of folic acid, 0.5 mg of calcium pantothenate, a suitable amount of an inorganic mixture, 1.75 mg of ferrous sulfate, 0.82 mg of zinc oxide, 25.3 mg of magnesium carbonate, 15 mg of potassium phosphate monobasic, 55 mg of potassium phosphate dibasic, 90 mg of potassium citrate, 100 mg of calcium carbonate, and 24.8 mg of magnesium chloride were mixed and prepared into granules, but may be prepared by modifying the mixture into various formulations according to use. Furthermore, the composition ratio of the vitamin and mineral mixture may be arbitrarily modified, and a health functional food may be prepared by mixing the aforementioned components by a typical health functional food preparation method.

### Preparation Example 5. Preparation of Health Functional Beverage

1 g of Compound 1 (Hex-AH-3′SL) of the present invention, 0.1 g of citric acid, 100 g of fructo oligosaccharide, and 900 g of purified water were mixed, and stirred, heated, filtered, sterilized, and cooled to prepare a beverage.

### INDUSTRIAL APPLICABILITY

The present invention shows high hemagglutination inhibitory activity and virus neutralizing activity so as to have excellent antiviral activity, thereby being effectively usable for preventing, ameliorating, or treating viral infectious diseases.

1. A compound comprising a multivalent sialyloligosaccharide residue, which is represented by the following Chemical Formula 1, an optical isomer thereof, or a pharmaceutically acceptable salt thereof: 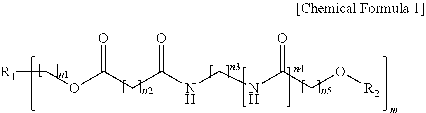
wherein in Chemical Formula 1,
R₁ is a C₄-C₆ cycloalkyl or a C₄-C₆ heterocycloalkyl,
R₂ is a sialyloligosaccharide,
n₁ is 0 or 1, n₂ is 1 or 2, n₃ is an integer from 2 to 10, n₄ is 0 or 1, n₅ is an integer from 1 to 8, and m is an integer from 1 to 6.

2. The compound, the optical isomer thereof, or the pharmaceutically acceptable salt thereof of claim 1,
wherein in Chemical Formula 1,
R₁ is a C₄-C₆ cycloalkyl or a C₄-C₆ heterocycloalkyl,
R₂ is a sialyl lactose,
n₁ is 0 or 1, n₂ is 1 or 2, n₃ is an integer from 2 to 10, n₄ is 0 or 1, and n₅ is an integer from 1 to 8, where the sum of n₃ and n₅ is an integer from 4 to 8, and
m is an integer from 4 to 6.

3. The compound, the optical isomer thereof, or the pharmaceutically acceptable salt thereof of claim 1,
wherein in Chemical Formula 1,
R₁ is a C₄ or C₆ cycloalkyl,
R₂ is a sialyl lactose,
n₁ is 0 or 1, n₂ is 1 or 2, n₃ is an integer from 2 to 10, n₄ is 0 or 1, and n₅ is an integer from 1 to 8, where the sum of n₃ and n₅ is an integer of 6 or 7, and
m is an integer of 4 or 6.

4. The compound, the optical isomer thereof, or the pharmaceutically acceptable salt thereof of claim 1, wherein the compound of Chemical Formula 1 is selected from the group consisting of the following structures of Chemical Formulae 2 to 7.

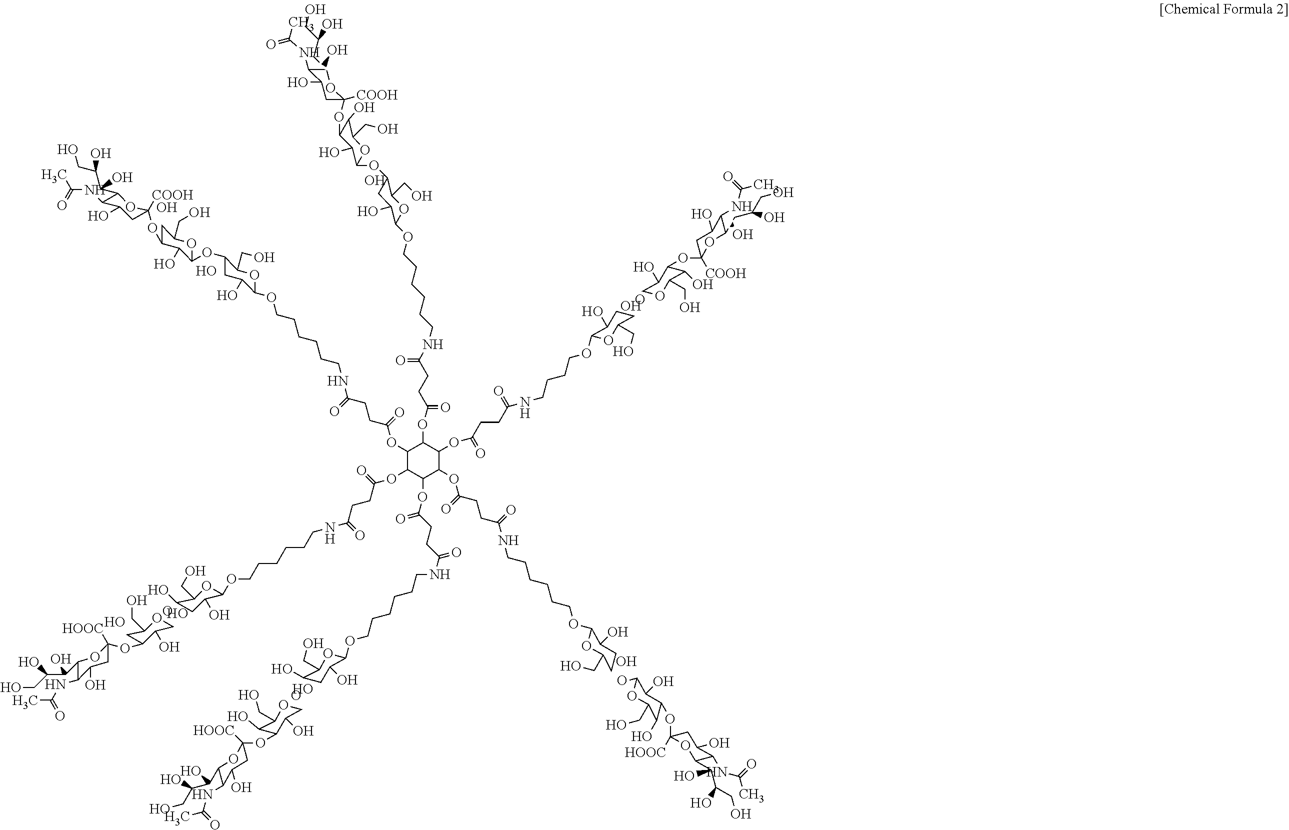

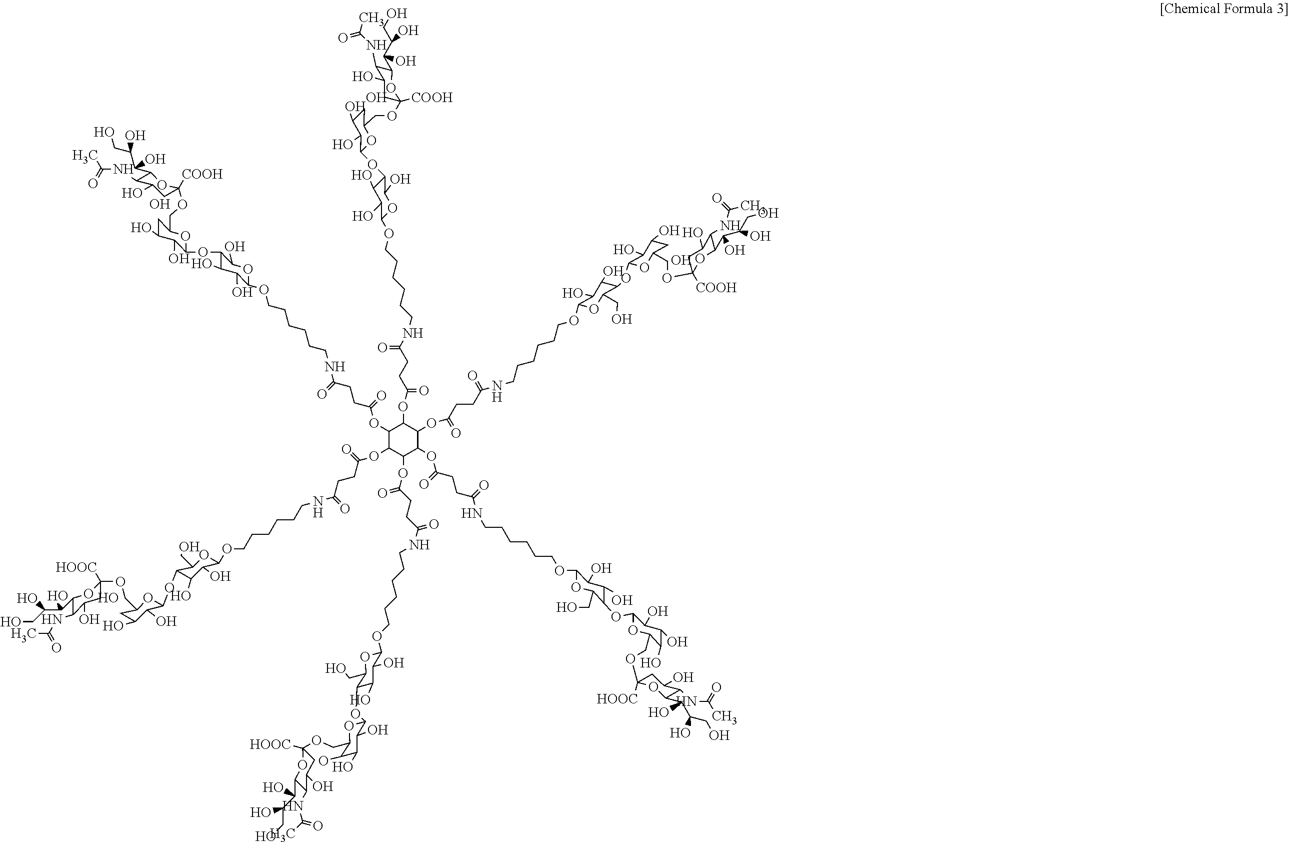

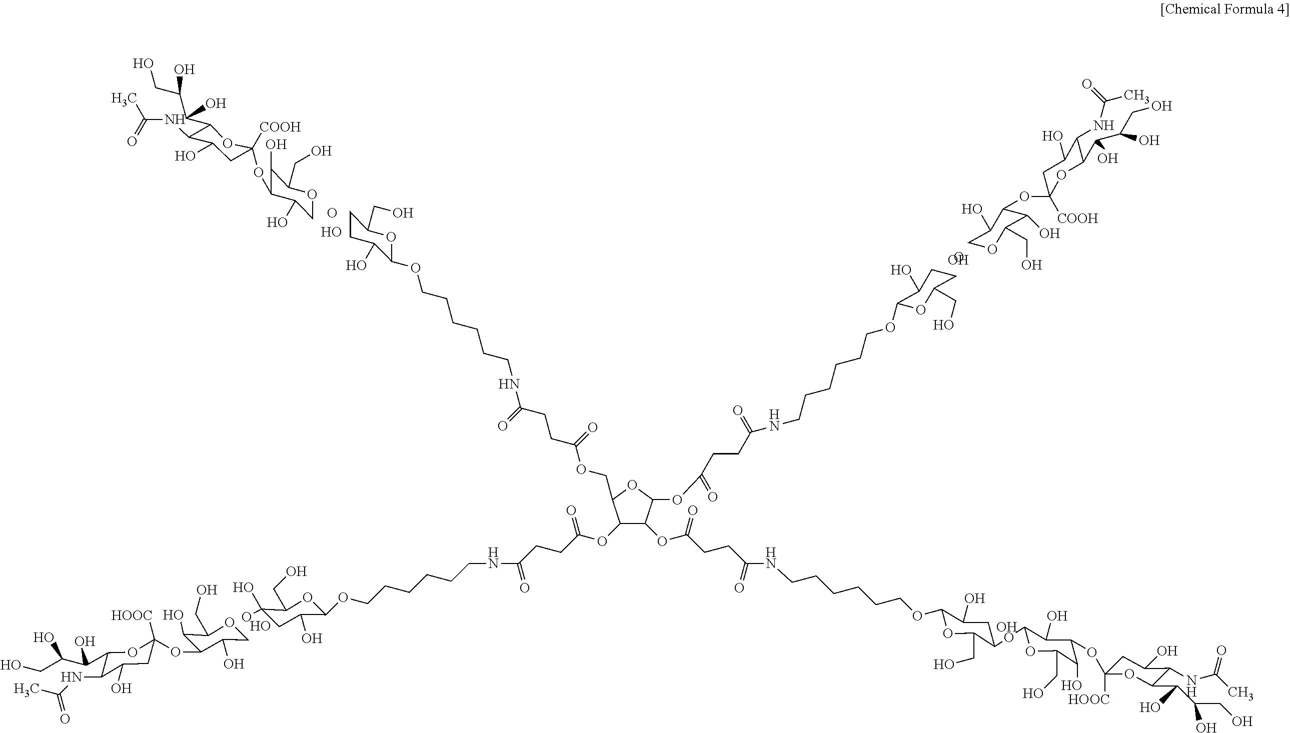

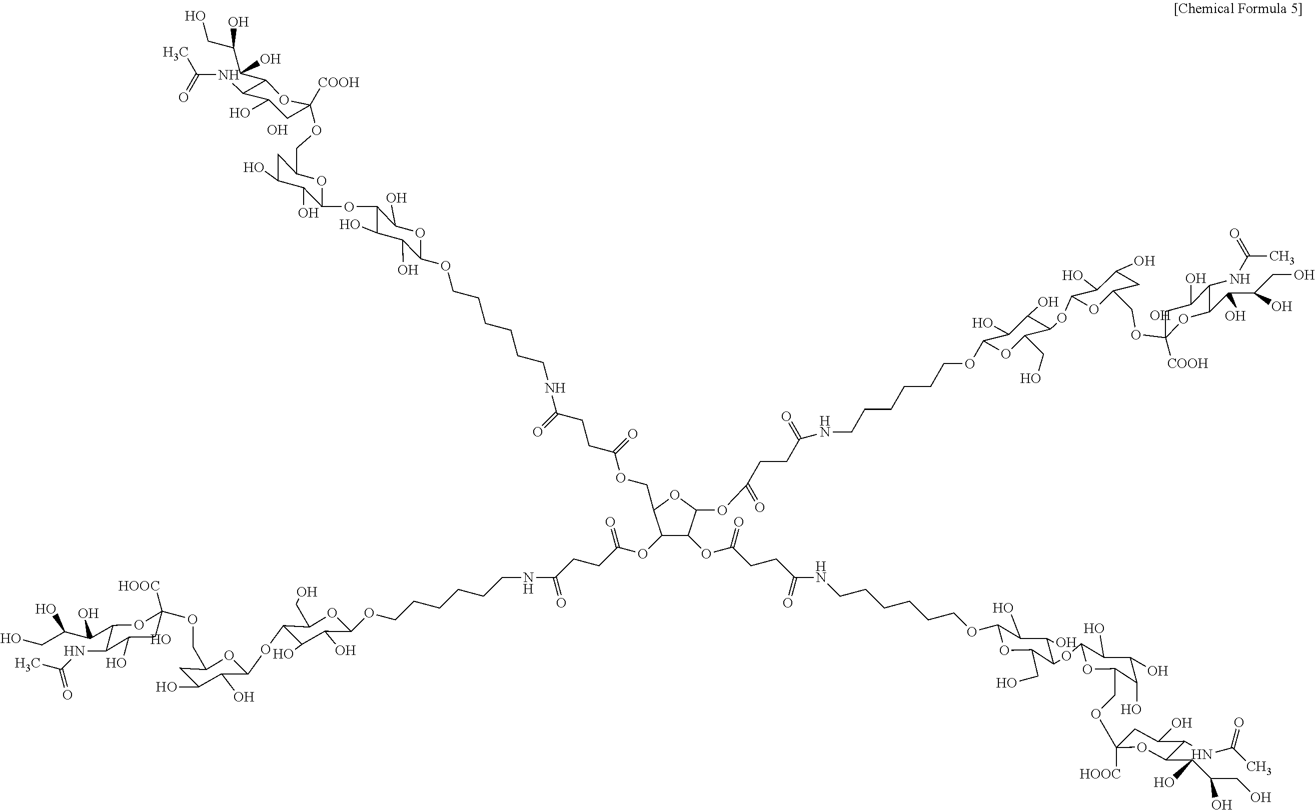

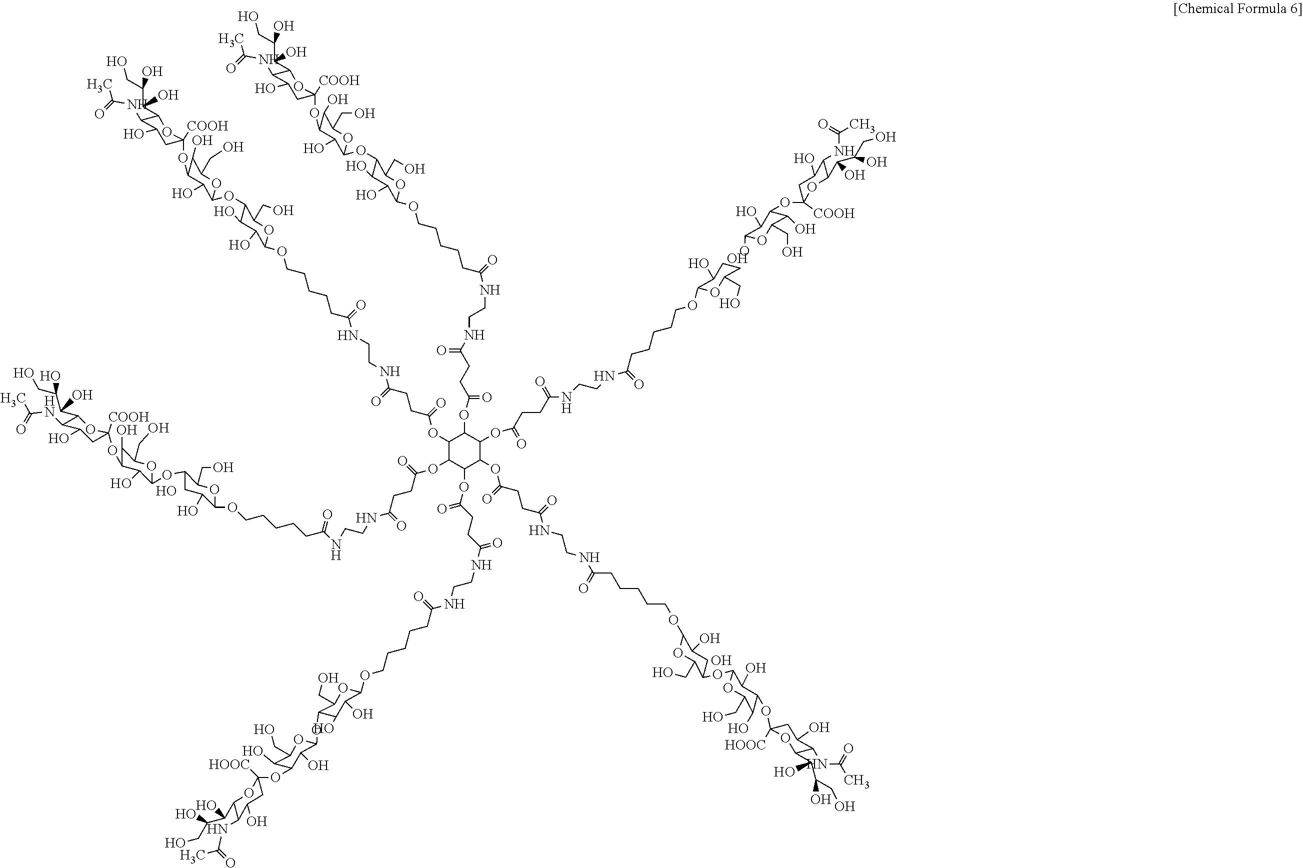

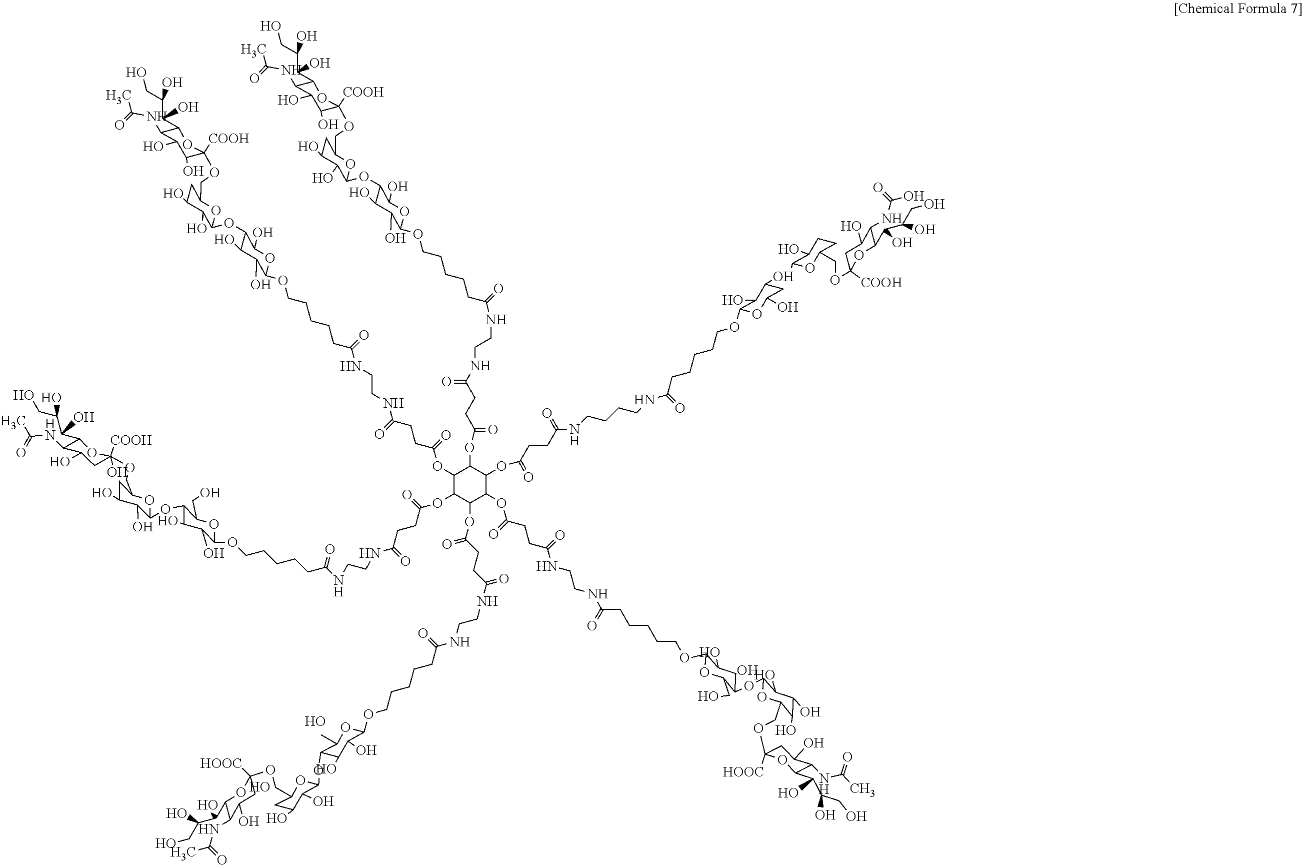

5. A composition comprising the compound comprising a multivalent sialyloligosaccharide residue, the optical isomer thereof, or the pharmaceutically acceptable salt thereof of claim 1 as an active ingredient for ameliorating or treating viral infectious diseases.

6. The composition of claim 5, wherein the virus is an influenza virus.

7. The composition of claim 6, wherein the influenza virus is influenza virus type A or influenza virus type B.

8. The composition of claim 7, wherein the influenza type A is one or more selected from the group consisting of H1N1, H3N2, and H9N2.

9. The composition of claim 7, wherein the viral infectious disease is the flu, the common cold, pharyngolaryngitis, bronchitis, pneumonia, avian flu, or swine flu.

10-13. (canceled)

14. A method for treating viral infectious diseases of an individual in need thereof, the method comprising: administering a therapeutically effective amount of the compound comprising a multivalent sialyloligosaccharide residue, the optical isomer thereof, or the pharmaceutically acceptable salt thereof of claim 1 to an individual.

15. (canceled)

16. The composition of claim 5, wherein the composition is one or more selected from the group consisting of a pharmaceutical composition, an animal drug composition, a health functional food composition, an animal feed additive composition, and a quasi-drug composition.
